(19) United States
(12) Patent Application Publication
(10) Pub. No.: US 2026/0137594 A1
(43) Pub. Date: May 21, 2026

(54) MEDICINE-PACK HOUSING AND MEDICATION SUPPORT APPARATUS

(71) Applicants: Takumi Hagiwara, Aichi (JP); Hirotaka Hayashi, Aichi (JP); Youhei Niitsuma, Kanagawa (JP); Yoshihide Sugiyama, Gifu (JP); Hiroshi Fujie, Aichi (JP); Manabu Yamanaka, Aichi (JP); Takato Mochizuki, Kanagawa (JP); Yu Yamaya, Gifu (JP); Sachika Tashiro, Kanagawa (JP); Terumitsu Azuma, Chiba (JP)

(72) Inventors: Takumi Hagiwara, Aichi (JP); Hirotaka Hayashi, Aichi (JP); Youhei Niitsuma, Kanagawa (JP); Yoshihide Sugiyama, Gifu (JP); Hiroshi Fujie, Aichi (JP); Manabu Yamanaka, Aichi (JP); Takato Mochizuki, Kanagawa (JP); Yu Yamaya, Gifu (JP); Sachika Tashiro, Kanagawa (JP); Terumitsu Azuma, Chiba (JP)

(73) Assignee: ETRIA Co., Ltd., Kanagawa (JP)

(21) Appl. No.: 19/390,610

(22) Filed: Nov. 16, 2025

(30) Foreign Application Priority Data
Nov. 21, 2024 (JP) 2024-203145

(51) Int. Cl. A61J 7/00; A61J 7/04

(52) U.S. Cl. CPC A61J 7/0084; A61J 7/0069; A61J 7/0454

(57) ABSTRACT

A medicine-pack housing includes a container in which a plurality of medicine packs are aligned in line and stored, a pickup device to pick up at least one of the plurality of medicine packs from the container, a mover to move in synchronization with a change in a length of the plurality of medicine packs in an aligned direction of the plurality of medicine packs due to a reduction in a remaining amount of the plurality of medicine packs inside the container, a sensor to detect a position of the mover, and processing circuitry to determine the remaining amount of the plurality of medicine packs inside the container based on a detection result of the sensor.

### CROSS-REFERENCE TO RELATED APPLICATION

This patent application is based on and claims priority pursuant to 35 U.S.C. § 119(a) to Japanese Patent Application No. 2024-203145, filed on Nov. 21, 2024, in the Japan Patent Office, the entire disclosure of which is hereby incorporated by reference herein.

### BACKGROUND

### Technical Field

The present disclosure relates to a medicine-pack housing and a medication support apparatus.

### Background Art

In medicine-pack housings, medicine packs are picked up by a pickup device from a container in which a plurality of medicine packs are aligned in line and stored.

Such medicine-pack housings may be medicine storage devices or medicine-pack housings provided with a medicine container in which, for example, a plurality of medicine packs, press through packs (PTP), and blister packs are stacked on top of one another and stored. Such devices measure the weight of the medicine container in which PTPs or blister packs are stored, and determine the remaining quantity such as the number of PTPs inside the medicine container, based on the measurement results.

### SUMMARY

The present disclosure described herein provides a medicine-pack housing and a medication support apparatus. The medicine-pack housing includes a container in which a plurality of medicine packs are aligned in line and stored, a pickup device to pick up at least one of the plurality of medicine packs from the container, a mover to move in synchronization with a change in a length of the plurality of medicine packs in an aligned direction of the plurality of medicine packs due to a reduction in a remaining amount of the plurality of medicine packs inside the container, a sensor to detect a position of the mover, and processing circuitry to determine the remaining amount of the plurality of medicine packs inside the container based on a detection result of the sensor. The medication support apparatus includes the medicine-pack housing, a pickup device to pick up at least one of the plurality of medicine packs from the container of the medicine-pack housing, and a conveyor to convey the at least one of the plurality of medicine packs picked up by the pickup device to a medicine tray.

### BRIEF DESCRIPTION OF THE DRAWINGS

A more complete appreciation of embodiments and the many attendant advantages thereof will be readily obtained as the same becomes better understood by reference to the following detailed description when considered in connection with the accompanying drawings.

FIG. 1A is a schematic front view of a medication support apparatus in its entirety.

FIG. 1B is a schematic side view of the medication support apparatus of FIG. 1A.

FIG. 2A is a vertical sectional view of a cartridge arranged in the medication support apparatus of FIG. 1A or FIG. 1B.

FIG. 2B is a bottom view of the cartridge of FIG. 2A.

FIG. 3 is a control block diagram illustrating a schematic control structure for a medication support apparatus.

FIG. 4A, FIG. 4B, FIG. 4C, FIG. 4D, FIG. 4E, and FIG. 4F are front views of a pickup device in the medication support apparatus of FIG. 1A or FIG. 1B, illustrating the progression of the operation of the pickup device.

FIG. 5A and FIG. 5B are front views of a pickup device in the medication support apparatus of FIG. 1A or FIG. 1B, illustrating the progression of the operation of the pickup device continued from FIG. 4F.

FIG. 6 is a flowchart of the sequence of basic operations of the medication support apparatus of FIG. 1A or FIG. 1B.

FIG. 7 is a flowchart of the processes of moving a home position (HP), which is a subroutine program of FIG. 6.

FIG. 8 is a supplemental flowchart of the operation of a pickup device in the medication support apparatus of FIG. 1A or FIG. 1B.

FIG. 9 is a diagram illustrating the control blocks of a medication support system.

FIG. 10 is an external perspective view of a medicine dispensing tray.

FIG. 11 is another external perspective view of a medicine dispensing tray.

FIG. 12A is a diagram illustrating a remaining-amount sensor when a feeler of a movable board is at an end position in a cartridge set in the medication support apparatus of FIG. 1A or FIG. 1B.

FIG. 12B is a diagram illustrating a remaining-amount sensor when the feeler of the movable board of FIG. 12A is at a near end position.

FIG. 13A is a diagram illustrating a remaining-amount sensor when the feeler of the movable board of FIG. 12A is at an end position.

FIG. 13B is a diagram illustrating a remaining-amount sensor when the feeler of the movable board of FIG. 12A is at a near end position.

FIG. 14 is a flowchart of remaining-amount determining processes when a remaining amount is detected.

FIG. 15 is a flowchart of a remaining-amount detecting operation at the time of initialization.

FIG. 16 is a flowchart of a remaining-amount detecting operation at the time of pack picking-up operation where a medicine pack is picked up from a cartridge.

FIG. 17 is a flowchart of basic operations of a medication support apparatus, including a remaining-amount detecting operation at the time of initialization as depicted in FIG. 15 and a remaining-amount detecting operation at the time of pack picking-up operation as depicted in FIG. 16.

FIG. 18A and FIG. 18B are diagrams each of which illustrates a configuration and operation of a sensor implemented by a transmissive optical sensor, according to a first modification.

FIG. 19 is a schematic perspective view of the transmissive optical sensor of FIG. 18A or FIG. 18B.

FIG. 20A and FIG. 20B are diagrams each of which illustrates a configuration and operation of a sensor implemented by a transmissive optical sensor, according to a second modification.

FIG. 21 is a flowchart of basic operations of a medication support apparatus, including one-pack thickness calculating processes, according to a third modification.

FIG. 22 is a flowchart of basic operations of a medication support apparatus, including a pickup retrying operation, according to a fourth modification.

FIG. 23 is a flowchart of the operations of FIG. 22 to which picking-out error processes are added.

FIG. 24 is a flowchart of basic operations of a medication support apparatus, including the operations to deal with an error in pickup due to multiple feeding, according to a fifth modification.

The accompanying drawings are intended to depict embodiments of the present disclosure and should not be interpreted to limit the scope thereof. The accompanying drawings are not to be considered as drawn to scale unless explicitly noted.

### DETAILED DESCRIPTION

The terminology used herein is for the purpose of describing particular embodiments only and is not intended to limit the present disclosure. As used herein, the singular forms “a”, “an”, and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

In describing embodiments illustrated in the drawings, specific terminology is employed for the sake of clarity. However, the present disclosure is not intended to be limited to the specific terminology so selected and it is to be understood that each specific element includes all technical equivalents that have the same structure, operate in a similar manner, and achieve a similar result.

Embodiments of the present disclosure are described in detail below with reference to the drawings. First, a medication support apparatus that makes up a medication support system is described.

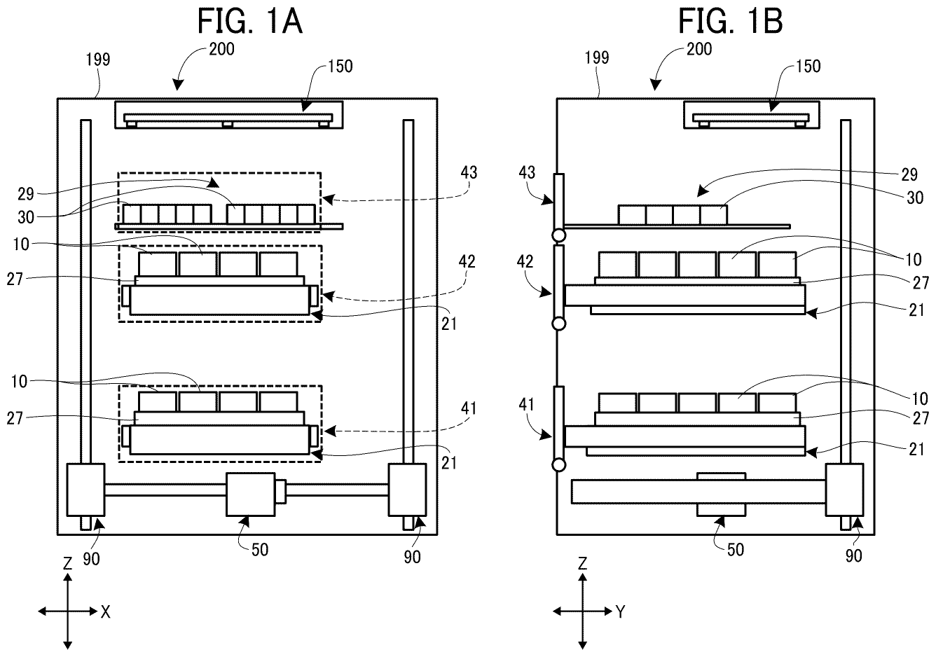

FIG. 1A is a schematic front view of a medication support apparatus in its entirety.

FIG. 1B is a schematic side view of the medication support apparatus 200 of FIG. 1A, according to the present comparative example.

As illustrated in FIG. 1A and FIG. 1B, the medication support apparatus 200 allows manual medicine-dispensing or medicine-sorting operations by, for example, nurse practitioners and care workers who work at a day-care center or nursing home to be switched to automatic medicine-dispensing or medicine-sorting operations by the medication support apparatus 200 itself.

The medication support apparatus 200 is provided with a plurality of cartridges 10 that are an example of medicine-pack housings, medicine dispensing trays 30 that are an example of medicine trays, a pickup device 50, a conveyor 90, and a first gate 41 to a third gate 43. A control board that contains a controller 150 as will be described later in detail is arranged at an upper portion of the medication support apparatus 200. The horizontal direction or the right and left directions of the medication support apparatus 200 of FIG. 1A is defined as an X-axis direction, where the horizontal direction or the right and left directions of the medication support apparatus 200 is also referred to as the width direction of the medication support apparatus 200. The front-rear direction or depth direction of the medication support apparatus 200 of FIG. 1B is defined as a Y-axis direction. The up and down directions or the orthogonal direction of the medication support apparatus 200 of FIG. 1A or FIG. 1B is defined as a Z-axis direction, where the up and down directions or the orthogonal direction of the medication support apparatus 200 is also referred to as the vertical direction.

Each one of the multiple cartridges 10 has a storage space 10a in which a plurality of medicine packages packing several kinds of medicines 3 are stored upon being stacked on top of each other in layers. The medicine package may be referred to simply as a pack or a medicine pack in the following description. The expression “stored upon being stacked on top of each other in layers” in the present disclosure indicates keeping the packs horizontally in such a manner that the front face is viewable.

The multiple cartridges 10 are arranged at the lowermost portion and middle portion of a housing 199 of the medication support apparatus 200 through a cartridge tray 27. In the case of the medication support apparatus 200 as illustrated in FIG. 1A and FIG. 1B, twenty cartridges 10 (4×5=20) are placed and held in a single cartridge tray 27. The cartridge tray 27 serves as a second container in which at least one of the multiple cartridges 10 is placed and held.

Each of the multiple cartridges 10 is placed and housed in a partitioned lattice-like side wall and bottom wall in the cartridge tray 27. On the bottom wall of the cartridge tray 27 corresponding to the cartridges 10, a rectangular through opening through which a pack can be picked up from below one of the multiple cartridges 10, making use of the elastic deformation of the pack as will be described later in detail, is formed. At least, each rectangular through opening on the bottom wall of the cartridge tray 27 has a bottom wall of thickness, size, and shape required to keep the packs inside the storage space 10a when the packs inside the storage space 10a are not to be taken out from a position below the cartridge 10.

Each one of the multiple medicine dispensing trays 30 is an example of a medicine dispensing apparatus, a medicine dispenser, or a medicine dispensing table in which the prescribed packs conveyed by the conveyor 90 are arranged. The medicine dispensing apparatus according to the present embodiment is used together with subdivision boxes 34 that serve as individual medicine dispensers and are detachable from the medicine dispensing tray 30. In the present embodiment described with reference to FIG. 1A and FIG. 1B, two medicine dispensing trays 30 each of which has twenty (=4×5) rooms 33 are arranged. The areas that include the medicine dispensing trays 30 where the packs are passed to the medicine dispensing tray 30 for automatic medicine dispensing are referred to as medicine dispensing areas 29 in the following description.

The pickup device 50 is an example of a pickup device that picks up specific one of the medicine packs from the storage space 10a of the cartridge 10. The conveyor 90 serves as a conveyor that conveys the pack picked up from the storage space 10a of one of the multiple cartridges 10 by the pickup device 50.

Each of the first gate 41 and the second gate 42 is an example of an entrance and exit gate for the container, which allows one of the multiple cartridges 10 to enter and exit the housing 199. The multiple cartridges 10 are inserted and set in the housing 199 through each one of the first gate 41 and the second gate 42. The open and close door of one of the first gate 41 and the second gate 42 are opened, and the drawer 21 in which the multiple cartridges 10 are aligned is drawn out to the front side. By so doing, the cartridges 10 are inserted or withdrawn.

The third gate 43 according to the present comparative example serves as an entrance and exit gate for the medicine dispenser, which allows one of the multiple medicine dispensing trays 30 to enter and exit the housing 199. The second gate 42 and the third gate 43 are arranged such that the packs can be taken out immediately after the packs are set or inserted into the medicine dispensing tray 30.

As illustrated in FIG. 1A, the medication support apparatus 200, two medicine dispensing trays 30 are arranged as described above, and as will be described later in detail, the medicine dispensing trays 30 are arranged for each time of medication such as a time in the morning, a time in the daytime, a time in the evening, and a time before going to bed. The third gate 43 for the medicine dispensing tray 30 is also arranged for each one of the medicine dispensing trays 30. Due to such a configuration, another one of the medicine dispensing trays 30 can be taken out even when the medicine dispensing operation is performed on a particular one of the multiple medicine dispensing trays 30.

The drawers 21 of the multiple cartridges 10 as illustrated in FIG. 1A and FIG. 1B are arranged in two stages on the upper and lower sides under the medicine dispensing trays 30 at the uppermost stage. However, no limitation is intended thereby, and all the drawers 21 may collectively be arranged on an upper side or may collectively be arranged on a lower side. Depending on the number of persons in the day-care center or nursing home, the cartridges 10 may be arranged in three stages to achieve similar advantageous effects.

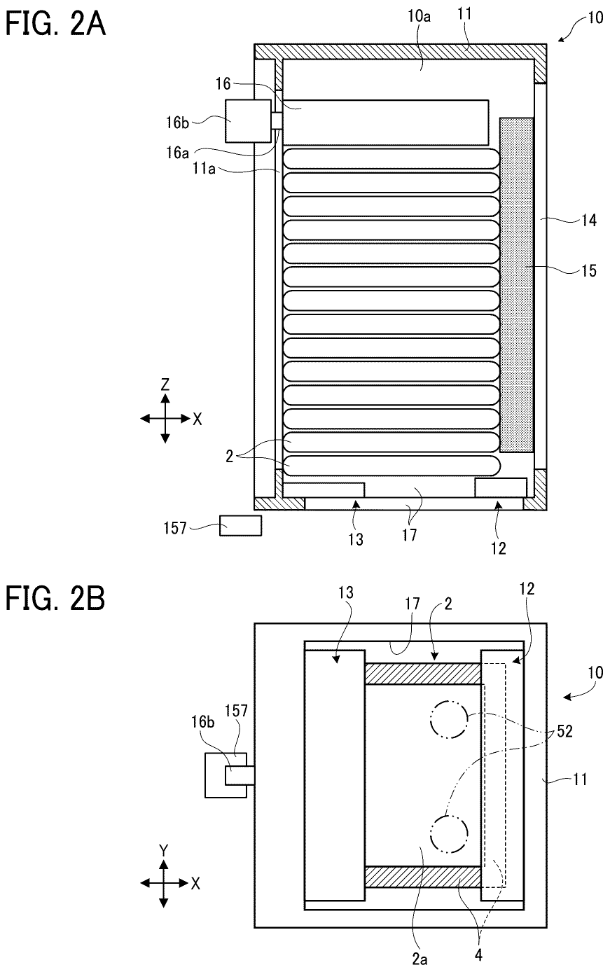

FIG. 2A is a vertical sectional view of the cartridge 10.

FIG. 2B is a bottom view of the cartridge 10 of FIG. 2A.

For example, each one of the multiple cartridges 10 includes a casing 11, a lid 14, a pack pickup slot 17, a movable board 16, a pack posture keeper 15, and a right support portion 12 and a left support portion 13 each of which serves as a support portion. The casing 11 makes up a container inside of which the storage space 10a is formed, and stores, for example, a plurality of medicine packs 2 or bound packages. In the following description, a single medicine pack 2 will represent such medicine packs 2 or bound packages. For example, the casing 11 is integrally or separately formed using resin. The lid 14 enables the multiple medicine packs 2 to be loaded or unloaded. The pack pickup slot 17 is formed in a lower portion or a bottom portion of the casing 11, and is used to pick up the medicine pack 2 in the storage space 10a of one of the multiple cartridges 10. Moreover, the pack pickup slot 17 allows the medicine pack 2, which is picked up from the storage space 10a by the pickup device 50 (see FIG. 1A and FIG. 1B), to pass therethrough.

The movable board 16 prevents the medicine pack 2 from falling out, and moves the lowermost one of the medicine packs 2 to a position close to the pack pickup slot 17 after the first one of the maximum number of medicine packs 2 that can be stored in the casing 11 is picked up. The pack posture keeper 15 keeps the posture of the medicine pack 2. The right support portion 12 and the left support portion 13 also support or hold the medicine pack 2 in the casing 11.

In the present embodiment, the to-be-picked-up portion of one of the multiple medicine packages 2 to be picked up from the storage space 10a of one of the multiple cartridges 10 by the pickup device 50 is at a lower portion or bottom portion of the storage space 10a of the corresponding one of the multiple cartridges 10. In other words, the to-be-picked-up portion includes the pack pickup slot 17, and the right support portion 12 and the left support portion 13 that serve as a pair of support portions or supporting member and support, at a plurality of points, one of the multiple medicine packs 2 to be picked up from the storage space 10a of one of the multiple cartridges 10.

When one of the multiple medicine packs 2 is picked up from the storage space 10a of one of the multiple cartridges 10 by the pickup device 50, the right support portion 12 and the left support portion 13 allow one of the multiple medicine packs 2 to pass through. On the other hand, when any one of the multiple medicine packs 2 is not to be picked up from the storage space 10a of one of the multiple cartridges 10, the multiple medicine packs 2 are to be stored and held in the casing 11. In order to achieve that, the right support portion 12 and the left support portion 13 are configured to restrict the passage of one of the multiple medicine packs 2.

As described above, the right support portion 12 and the left support portion 13 serve as a pair of support portions that support or hold the multiple medicine packs 2 in the storage space 10a of the one of the multiple cartridges 10, and each one of the right support portion 12 and the left support portion 13 is fixed and immovable such that one of the multiple medicine packs 2 can be picked up in a stable manner from the storage space 10a of one of the multiple cartridges 10 by the pickup device 50. The right support portion 12 and the left support portion 13 are a pair of fixation members that are fixed or attached to the pair of bottom-wall inner surfaces of the right bottom-wall edge and left bottom-wall edge of the pack pickup slot 17, respectively. The pack pickup slot 17 has a function to allow the pair of suction pads 52 of the pickup device 50 illustrated in FIG. 2B, which serves as an air suction unit or a suction unit, to pass therethrough in order to pick up one of the multiple medicine packs 2 and a function to allow the picked-up medicine pack 2 and the pair of suction pads 52 to pass therethrough.

In one of the multiple cartridges 10 illustrated in FIG. 2B, a pair of positions at which the pair of suction pads 52 adsorb or suck up one of the multiple medicine packs 2 stored in the storage space 10a of one of the multiple cartridges 10, are indicated by a pair of ring-shaped dot-dot-dash lines. Such a pair of positions may be referred to as a pair of suction-pad positions in the following description. The right support portion 12 and the left support portion 13 that are arranged underneath the storage space 10a of one of the multiple cartridges 10 support the multiple medicine packs 2 in the storage space 10a of one of the multiple cartridges 10 such that the multiple medicine packs 2 in one of the multiple cartridges 10 do not fall from the pack pickup slot 17. As will be described later in detail in regard to the operation of the pickup device 50, when the medicine pack 2 at the bottom of the storage space 10a inside the cartridge 10 is sucked and picked up by the pair of suction pads 52, one of the multiple medicine packs 2 is sucked by the pair of suction pads 52 at two suction-pad positions in the Y-axis direction near both ends of the right support portion 12. When the lowermost one of the multiple medicine packs 2 is picked up from the storage space 10a of one of the multiple cartridges 10 by the pair of suction pads 52, the pair of suction pads 52 passe near both ends of the right support portion 12 in the Y-axis direction to suck and hold one of the multiple medicine packs 2.

As illustrated in FIG. 2B, two points at which the pair of suction pads 52 contact the medicine package are arranged near both ends of the right support portion 12 in the Y-axis direction. Due to such a configuration, the possibility of an error in which the suction by the pair of suction pads 52 is disabled can be avoided and prevented, and the picking up operation can be successfully done. In other words, the medicine pack 2 is sucked and adhered to the pair of suction pads 52 at both ends in the Y-axis direction, and the bag 2a of the medicine pack 2, which is made of film, is stretched and can withstand the deformation. Accordingly, both reliable support or holding of the multiple medicine packs 2 in the storage space 10a of one of the multiple cartridges 10 and smooth removal of one of the multiple medicine packs 2 can be achieved.

The pack posture keeper 15 is formed of sponge rubber having appropriate elasticity. The movable board 16 as well as the shaft 16a and the feeler 16b are made of, for example, resin or metal. The pack posture keeper 15 and the movable board 16 hold the posture of the multiple medicine packs 2 under normal operating conditions in the casing 11. As explicitly illustrated in FIG. 2A, the posture of the multiple medicine packs 2 is held orderly in the Z-axis direction in a substantially horizontal state. In order to achieve the above-described function, the movable board 16 is arranged to move at least one of the multiple medicine packs 2 left in the casing 11 toward the pack pickup slot 17 as moving downward in the casing 11 in the Z-axis direction by its own weight.

As illustrated in FIG. 2A, a long groove 11a that extends in the Z-axis direction with a predetermined width in the X-axis direction is formed in the side wall of the casing 11. The shaft 16a with the feeler 16b, which is an example of a mover, is arranged at one end of the movable board 16 so as to protrude from the long groove 11a. As the shaft 16a of the movable board 16 with the feeler 16b is guided in the Z-axis direction parallel to the long groove 11a, the posture of the multiple medicine packs 2 can be held in the Z-axis direction. In FIG. 2A, the multiple medicine packs 2 in the storage space 10a of one of the multiple cartridges 10 are stacked on top of each other in layers in a substantially horizontal state. However, in actuality, left portions of the medicine packs 2 in FIG. 2A filled with the medicines 3 are expanding.

A set of the multiple medicine packs 2 are orderly stored in the casing 11 toward the upper side from the pack pickup slot 17 on the right support portion 12 and the left support portion 13. The timing at which the multiple medicine packs 2 are supplied into the storage space 10a of one of the multiple cartridges 10 may be, for example, the timing at which a patient who takes medicines or occupant is examined in a day-care center or nursing home, which is typically once a two weeks, or the timing at which the multiple medicine packs 2 in the storage space 10a of one of the multiple cartridges 10 run out. When some medicine packs 2 are left in the storage space 10a of one of the multiple cartridges 10 at the time of replenishment, such replenishment is continued from the back of the remaining medicine packs 2. Under normal operating conditions, the above-described setting of the multiple medicine packs 2 into the storage space 10a of one of the multiple cartridges 10 and the replenishment of the multiple medicine packs 2 are done by a staff or the like in a day-care center or nursing home. However, no limitation is indicated thereby. In particular, in configurations where cartridges are used instead of containers and the setting or replenishment of the medicine packs 2 is done automatically, the setting or replenishment of the medicine packs 2 may be done differently.

The lid 14 is used by a staff or the like who works at a day-care center or nursing home to enable loading or unloading of the multiple medicine packs 2 stored in the storage space 10a of one of the multiple cartridges 10, and as illustrated in FIG. 2A, is formed with a wide length in the Z-axis direction of the casing 11 and has a predetermined opening width.

As illustrated in FIG. 2A, the types of the multiple medicine packs 2 in the storage space 10a of one of the multiple cartridges 10 are sorted or divided based on the timings at which medicines are taken. For example, one cartridge contains medicines to be taken in fourteen days in the morning by Mr. or Ms. A. In view of the above circumstances, if Mr. or Ms. A takes the medicines not only in the morning but also in the daytime or in the evening, or before going to bed, four cartridges 10 are required in total. However, no limitation is indicated thereby. For example, in an alternative embodiment of the present disclosure, a single cartridge 10 may be prepared for each person or patient who takes medicines. In such cases, for example, a pack to be used in the morning on day one, a pack to be used at lunchtime on day one, a pack to be used in the evening on day one, a pack to be used before going to bed on day one, a pack to be used in the morning on day two, a pack to be used at lunchtime on day two, and a pack to be used in the evening on day two may be stored in the order listed upward from the pack pickup slot 17 parallel to the direction in which packs are taken out from the multiple cartridges 10.

In the present embodiment, the right support portion 12 and the left support portion 13 are fixed to the bottom-wall inner surface of the pack pickup slot 17 of the casing 11 such that one of the multiple medicine packs 2 can be picked up in a steadily stable manner from the storage space 10a of one of the multiple cartridges 10 by the pickup device 50. In other words, the right support portion 12 and the left support portion 13 are fixed when the right support portion 12 and the left support portion 13 hold both ends of the medicine pack such as the medicine pack 2 or the bound packages around the pack pickup slot 17 arranged at the lower portion of one of the multiple cartridges 10.

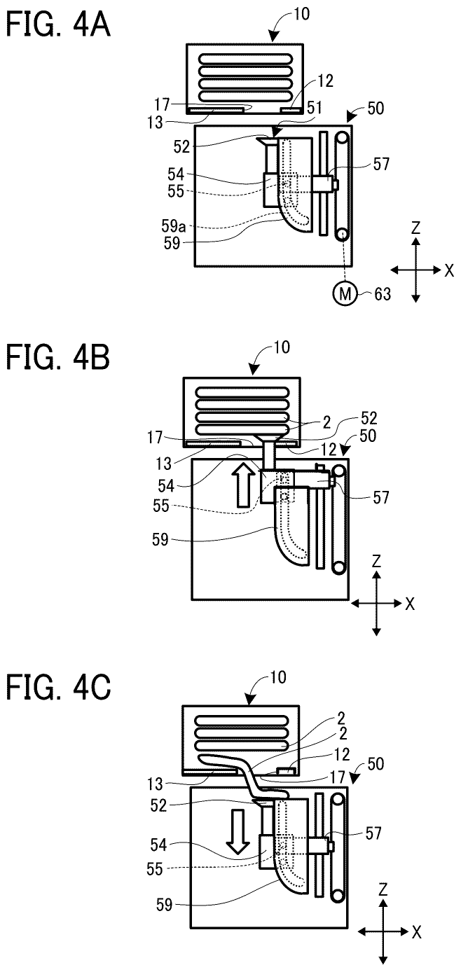

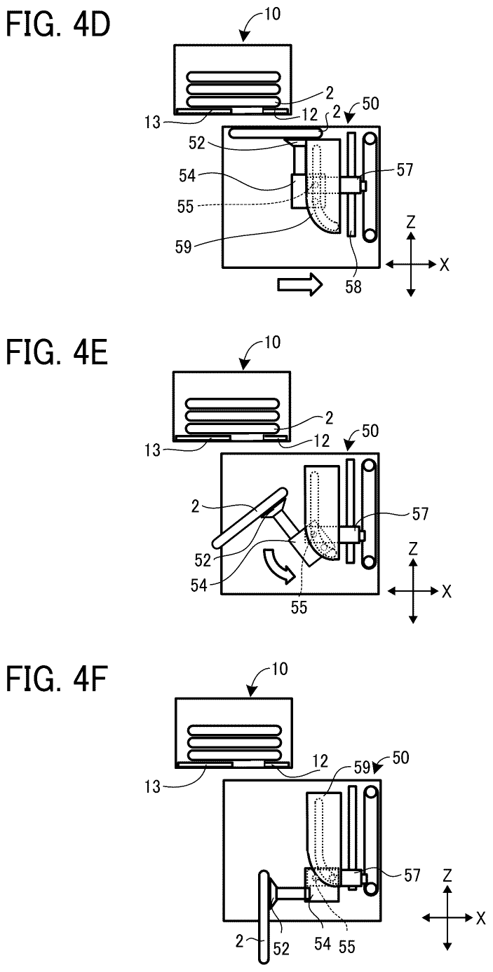

The medicine pack 2 that is stored in the storage space 10a of the cartridge 10 is supported by a right support portion 12 and a left support portion 13 so as not to drop. The right support portion 12 supports a right end where the medicine pack 2 is sucked up, and the left support portion 13 supports a left end on the other side. The right support portion 12 and the left support portion 13 are different in the length of the portion that supports the multiple medicine packs 2, and the length of the portion of the right support portion 12 that supports the multiple medicine packs 2 is shorter than that of the left support portion 13. As will be described later in detail with reference to FIG. 4C, when the lowermost one of the multiple medicine packs 2 stored in the storage space 10a of the cartridge 10 is adsorbed or sucked by the pair of suction pads 52 and is pulled out from the storage space 10a of the cartridge 10, the medicine pack is bent due to its elasticity. Accordingly, the medicine pack can easily be pulled out.

In the above configuration, the pair of support portions including the right support portion 12 and the left support portion 13 are of fixed type. Accordingly, the front end of the next one of the multiple medicine packs 2 to be taken out can be reliably held and does not jump out or fall off together with one of the multiple medicine packs 2 that is being taken out. As the pair of support portions including the right support portion 12 and the left support portion 13 do not swing or rotate, the medicine pack 2 are held with high stability with no deformation or the like due to unintentional nipping or pressing in the returning operation.

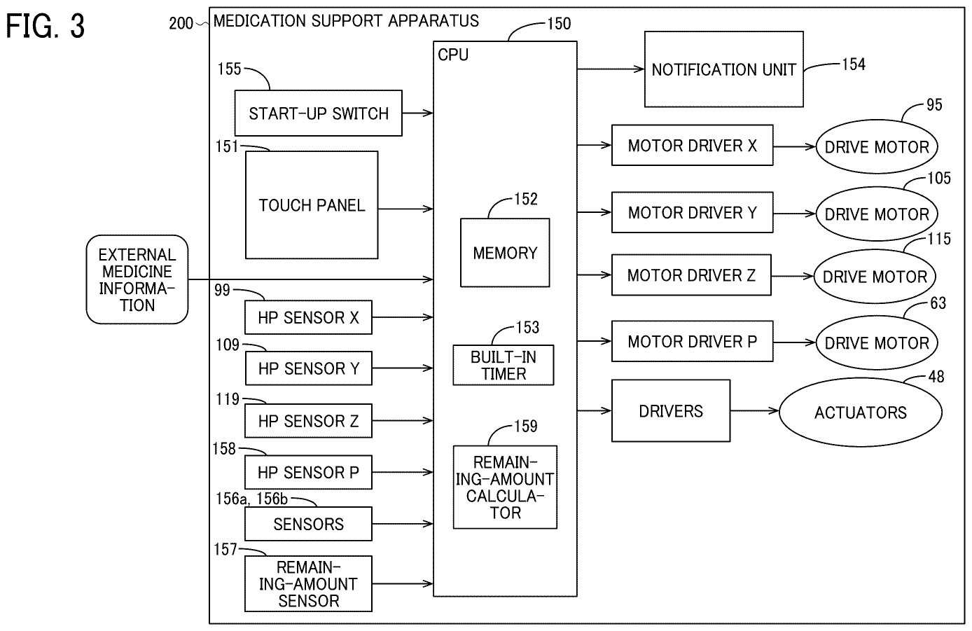

FIG. 3 is a diagram illustrating the control blocks of a medication support apparatus.

As illustrated in FIG. 3, the medication support apparatus 200 includes a central processing unit (CPU) that serves as a controller 150 that controls the operation of, for example, the components or elements of the medication support apparatus 200. For example, the CPU may be provided with a built-in memory 152 or a built-in timer 153, and serves as the remaining-amount calculator 159 that is an example of a remaining-amount determination unit. The CPU according to the present embodiment may provide notification to, for example, a user including staff or the like at a timing consistent with the program or may instruct the medication support apparatus 200 to perform a particular operation, based on various kinds of input such as the inputs from a sensor as will be described later in detail. To the built-in memory 152, prescription data or medicine information is externally input as external medicine information.

The CPU may have, for example, a computing or control function, and a timer or clocking function. A memory 152 includes, for example, a read-only memory (ROM), a random-access memory (RAM), and an external memory. The ROM according to the present embodiment stores, for example, a program readable by the above CPU and various kinds of data in advance. Such a program stored in the ROM may be a program used in the flowchart of the controlling processes as will be described later in detail. The above various kinds of data may be, for example, the data about the relation between the medicine packs 2 and the rooms 33 or the subdivision boxes 34 of one of the medicine dispensing trays 30 allocated to each of the patients who take medicines, the data about the relation between the medicine packs 2 and the multiple rooms 33 or the multiple subdivision boxes 34 of one of the medicine dispensing trays 30 assigned to each one of the times of medication, or the data about the relation between the medicine packs 2 and the rooms 33 or the subdivision boxes 34 of one of the medicine dispensing trays 30 sorted according to the order in which medicines are to be taken.

The CPU has an input and output (I/O) port, and a touch panel 151 that is an example of a user interface (UI) provided with an input unit and a display unit is electrically connected to that input and output port of the CPU. Through the touch panel 151, various kinds of inputs can be made, and for example, the current time, the progress of the storing process of packs, or the stop time are displayed on the touch panel 151. How such inputs are made or the structure or configuration of such a display unit are not limited thereto, and may be, for example, an input unit and a display unit may be arranged separately. Alternatively, a combination of an input device and a display interface such as a combination of a keyboard and a light-emitting diode (LED) display may be adopted.

To the input port of the CPU, a start-up switch 155 that drives the medication support apparatus 200 to operate is electrically connected. Once the start-up switch 155 is touched or pressed down, the medicine dispensing operation to the medicine dispensing trays 30 starts step by step. The medicine dispensing operation to the medicine dispensing trays 30 may start at a time determined in advance by the built-in timer 153.

The CPU according to the present embodiment has an input port, various types of sensors such as a medicine dispensing tray sensor 156a that detects the type of medicine dispensing tray 30 stored in the medication support apparatus 200 or determines whether or not there is any medicine dispensing tray 30, a container sensor 156b that determines whether or not there is any cartridge 10, and a remaining-amount sensor 157 that is an example of a sensor to detect the position of the feeler 16b attached to the movable board 16 of the cartridge 10 are electrically connected to the input port of the CPU. The medicine dispensing tray sensor 156a and the container sensor 156b are illustrated in FIG. 3.

To the input port of the above CPU, a home position (HP) sensor 99 that is used for a home position (HP) sensor X to detect the home position (HP) of an X-axis direction conveyor in the pickup device 50, an HP sensor 109 that is used for an HP sensor Y to detect the home position (HP) of a Y-axis direction conveyor in the pickup device 50, and an HP sensor 119 that is used for an HP sensor Z to detect the home position (HP) of a Z-axis direction conveyor in the pickup device 50 are electrically connected.

Further, to the input port of the CPU, a home position (HP) sensor 158 for an HP sensor P that detects the home position (HP) of the suction pad 52 of the suction device 51 in the pickup device 50 is electrically connected.

To the output port of the above CPU, the drive motor 95 for the X-axis direction conveyor, the drive motor 105 for the Y-axis direction conveyor, the drive motor 115 for the Z-axis direction conveyor, and the drive motor 63 used to change the posture or attitude of the pair of suction pads 52 are electrically connected to the input port of the CPU through various kinds of motor drivers X, Y, Z, and P, respectively.

To the output port of the CPU, a suction pump 48 that serves as various kinds of actuator is electrically connected through various kinds of drivers. To the output port of the CPU, a notifier may be electrically connected. Such a notifier reports what sort of state or conditions the components or elements of the medication support apparatus 200 are in by means of, for example, the light emitted from a light-emitting diode (LED) and the sound or vibration including voice. Moreover, such a notifier may be provided with, for example, a loudspeaker or a light that indicates that the medicines are to be taken so that the staff or the like away from the medication support apparatus 200 can be notified of such a time of medication.

The external medicine information is also input to the CPU through an input and output (I/O) interface, and is stored in the built-in memory 152. For example, the external medicine information is used for the allocation of medicines to patients who take medicines. For example, the LEDs of the drawer 21 may be electrically connected to each other.

Once the input data from the touch panel 151 and various kinds of signals from various types of sensors or the HP sensors 99, 109, 119, and 158 are input to the CPU, a command signal is newly output from the CPU. In other words, the CPU according to the present embodiment outputs a command signal used to control the audio device or the optical device of the display device of the touch panel 151 including the above notifier, the LEDs, the suction pump 48, the drive motor 63, the drive motor 95, the drive motor 105, the drive motor 115, or the multiple drives that correspond to the multiple LEDs.

The HP sensor 158 for the HP sensor P and the drive motor 63 that outputs power through the motor driver P are used to control or drive the mechanism for moving the suction device to move upward and downward. The CPU according to the present embodiment has a function to execute the control operation as will be described later in detail in the following description or the flowchart of the controlling processes.

For example, it is desired that the drive motor 63, the drive motor 95, the drive motor 105, and the drive motor 115 be a stepper motor driven by pulse input from the viewpoint of its control with a high degree of precision and precise amounts of movement.

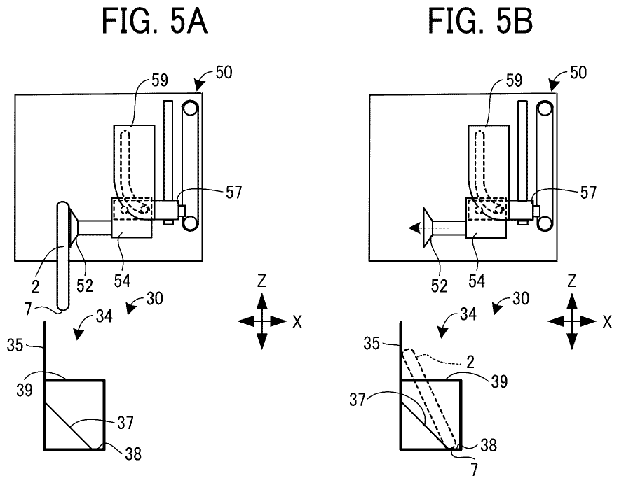

FIG. 4A to FIG. 5B are front views of a pickup device, illustrating the progression of the operation of the pickup device.

For the sake of explanatory convenience, it is assumed in the present embodiment that, due to the operation of the conveyor 90 illustrated in FIG. 1A and FIG. 1B, the pickup device 50 is arranged below the cartridge 10 provided for one of the drawers 21 arranged at the central portion of the housing 199 illustrated in FIG. 1A and FIG. 1B. After the pair of suction pads 52 of the pickup device 50 sucks and pulls out the lowermost pack in the storage space 10a of the cartridge 10, as the conveyor 90 operates, the pickup device 50 moves above the medicine dispensing tray 30, which is placed uppermost in FIG. 1A and FIG. 1B. Then, the pickup device 50 automatically drops and insert the pack into the subdivision box 34 attached to a specific room of the medicine dispensing tray 30. In FIG. 4A to FIG. 5B, the cartridges illustrated in FIG. 2A and FIG. 2B are used as the cartridges 10.

As illustrated in FIG. 4A, the pickup device 50 is moved under one of the multiple cartridges 10 by the operation made by the conveyor 90 illustrated in FIG. 1A and FIG. 1B, and stops moving. Under such conditions, the drive motor 63 of a suction-device lifting and lowering unit is stopped, and the pair of suction pads 52 are positioned under the position of the top face of the pickup device, which indicates the position of the top face of the picking-up frame of the pickup device 50 in a housing.

Subsequently, as described with reference to FIG. 4B, the drive motor 63 is driven to rotate, and firstly, the suction device base 57 and the pair of suction pads 52 move upward in a straight line through the coupling between the suction device base 57 that linearly moves and the suction-pad supporting member 54 that linearly and rotationally moves. Accordingly, the pair of suction pads 52 enter from the pack pickup slot 17 between the right support portion 12 and the left support portion 13, and as soon as the pair of suction pads 52 contact one of the multiple medicine packs 2 at the bottom of the storage space 10a of one of the multiple cartridges 10, the pair of suction pads 52 suck that medicine pack 2. In so doing, the suction pump 48 as described above is driven to operate in advance so that the suctioning operation can be performed.

Subsequently, as illustrated in FIG. 4C, as the drive motor 63 is reversely operated, the pair of suction pads 52 move downward while sucking one of the multiple medicine packs 2 by the pair of suction pads 52, and the front end of one of the multiple medicine packs 2 is pulled out from the storage space 10a of one of the multiple cartridges 10. The front end of the medicine package indicates a side of the medicine package to be absorbed or sucked up by the pair of suction pads 52, and the same applies in the following description. The operation of pulling out the front end of one of the multiple medicine packs 2 from the pack pickup slot 17 of one of the multiple cartridges 10 results in sort of free deformation in the pulled-out medicine pack 2.

Subsequently, as illustrated in FIG. 4D, by the operation of the conveyor 90 (see FIG. 1A and FIG. 1B), the pickup device 50 is moved in the X-axis direction, which is the lateral direction, and the rear end of one of the multiple medicine packs 2 is drawn out or picked up from the storage space 10a of one of the multiple cartridges 10. Immediately after that, as illustrated in FIG. 4E and FIG. 4F, by the operation of the drive motor 63, one of the multiple medicine packs 2, which is approximately in a horizontal state and is sucked and held by the pair of suction pads 52, is rotated by substantially 90 degrees to change the posture to an approximately vertical or upright posture. In such rotational operation, the rotary shaft 55 that is arranged on the suction-pad supporting member 54 moves along the guide groove 59a of the guide 59. Accordingly, the posture of one of the multiple medicine packs 2 can be changed from an approximately horizontal posture to an approximately vertical posture. The above series of operation in the above configuration may be achieved by a series of operations performed by a single drive motor 63.

Subsequently, as illustrated in FIG. 5A, the pickup device 50 that includes the pair of suction pads 52 and vertically holds one of the multiple medicine packs 2 is carried to the medicine dispensing area 29 substantially directly above the medicine dispensing trays 30 by the conveyor 90, and the operation of the suction pump 48 is terminated. As illustrated in FIG. 5B, when the suction holding of one of the multiple medicine packs 2 by the pair of suction pads 52 is to be released, so-called vacuum breakdown in which negative pressure is changed to atmospheric pressure is performed by ejecting a small amount of air using the above suction pump 48 that serves as an air supply unit or compressor. As a result, the suction holding of one of the multiple medicine packages 2 by the pair of suction pads 52 is released, and one of the multiple medicine packages 2 indicated by broken line is inserted into an opening 39 of desired one of the multiple subdivision boxes 34 in one of the rooms 33 of the medicine dispensing tray 30.

Subsequently, the bottom end 7 of the inserted medicine pack 2 contacts the inclined portion 37, and simply slides down along the inclined portion 37. In so doing, the medicine pack 2 is stored further inside the subdivision box 34 while the top end of the medicine pack 2 is being tilted backward along a rear side 35. As illustrated in FIG. 5B, the bottom end 7 of the inserted medicine pack 2 contacts a bottom side 38 of the subdivision box 34, and the medicine pack 2 stops moving. Accordingly, a surface of the medicine pack 2 is oriented upward, and medication-related information can be checked as desired.

As described above, in the present embodiment, when desired one of the multiple medicine packs 2 is picked up from one of the multiple cartridges 10, the pickup device 50 is positioned or arranged under one of the multiple cartridges 10, and such desired one of the multiple medicine packs 2 is taken out in the downward direction of the storage space 10a of one of the multiple cartridges 10. When one of the multiple medicine packs 2 is taken out from the lower side of one of the multiple cartridges 10 as described above, the next one of the multiple medicine packs 2 automatically moves downward or toward the pack pickup slot 17 due to the self weight of the movable board 16 and the multiple medicine packs 2 left in the storage space 10a of the cartridge 10. Due to such a configuration, the pickup device 50 can perform the same operation with a relatively simple configuration regardless of the number of the multiple medicine packs 2 left in the storage space 10a of the cartridge 10.

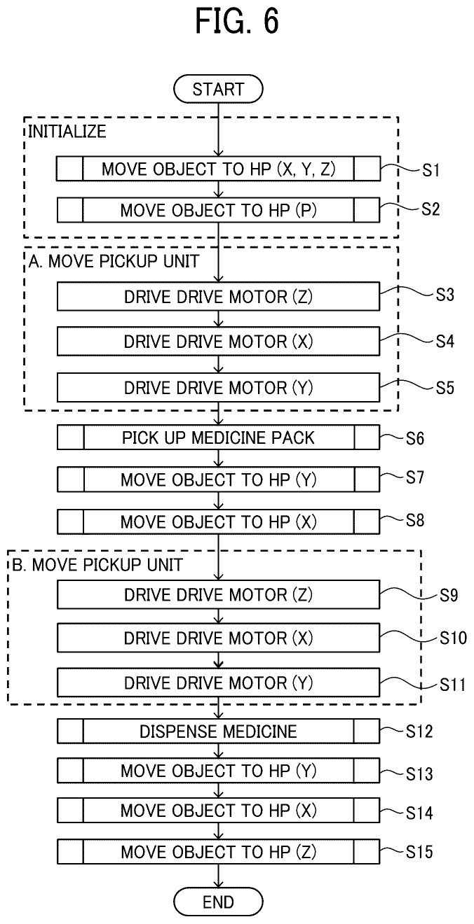

FIG. 6 is a flowchart of the sequence of basic operation of the medication support apparatus 200.

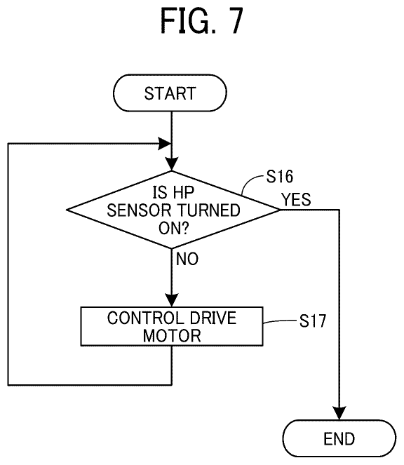

FIG. 7 is a flowchart of the processes of moving the home position (HP), which is a subroutine program of FIG. 6.

The operations in FIG. 6 and FIG. 7 are executed under the control commands from the CPU of the controller 150. When the start-up switch 155 illustrated in FIG. 3 is turned on, the process starts, and in steps S1 and S2, firstly, initialization is performed as illustrated in FIG. 6 (see the initialization part indicated by a frame of broken line in FIG. 6).

In the initialization, the HP sensor and the corresponding drive motor are controlled by the subroutines as depicted in FIG. 7, and an object to be controlled is moved to a predetermined home position (HP) where the HP sensor is turned on (see steps S16 to S17 in FIG. 7). A series of processes are automatically performed by a built-in timer 153 as depicted in FIG. 3 as follows at prescribed timings that correspond to the medication support operations in the morning, at lunchtime, in the evening, or before going to bed, based on the medicine information that is externally obtained in advance or the input from the touch panel 151 illustrated in FIG. 3.

At predetermined times, in steps S3 to S5, the CPU drives the drive motor 115, the drive motor 95, and the drive motor 105 that correspond to Z, X, and Y, respectively, in the order listed to move the pickup device 50 to desired one of the cartridges 10 (see “A. MOVE PICKUP DEVICE” indicated by a frame of broken line in FIG. 6). Subsequently, as a series of picking-up operations, a drive motor 63 that is used for a drive motor P is driven so that the pair of suction pads 52 are moved close to the medicine pack 2 (see FIG. 4B), and the vacuum ejector valve is controlled to make the pair of suction pads 52 suck the medicine package 2. Subsequently, the drive motor 63 and a drive motor 95 that is used for a drive motor X are operated in cooperation with each other to pick up one of the medicine packs 2 from a position below one of the multiple cartridges 10 (see FIG. 4C and FIG. 4D). Then, in steps S6 to S8, the posture of the pair of suction pads 52 are rotated, and the medicine pack 2 is vertically held (see FIG. 4E and FIG. 4F).

Subsequently, in steps S9 to S11, the CPU drives the drive motor 115, the drive motor 95, and the drive motor 105 that correspond to Z, X, and Y, respectively, in the order listed to move the pickup device 50 so as to move of the medicine pack 2 to a position above desired one of the rooms 33 of one of the medicine dispensing trays 30 (see “B. MOVE PICKUP DEVICE” indicated by a frame of broken line in FIG. 6). Then, medicines are dispensed. In the medicine dispensing operation, after the operation of the pickup device 50 is terminated, the vacuum ejector valve is controlled to cause vacuum breakdown. As a result, suctioning is released to separate one of the medicine packs 2 and dispense the separated medicine package to the medicine dispensing tray 30 (see FIG. 5A and FIG. 5B). These operations are performed several times. When the delivery of the medicine packs for a predetermined number of persons is completed, the loudspeaker or the light of the notifier 154 is instructed to notify a medication assistant that the preparation is complete (see steps S12 to S15).

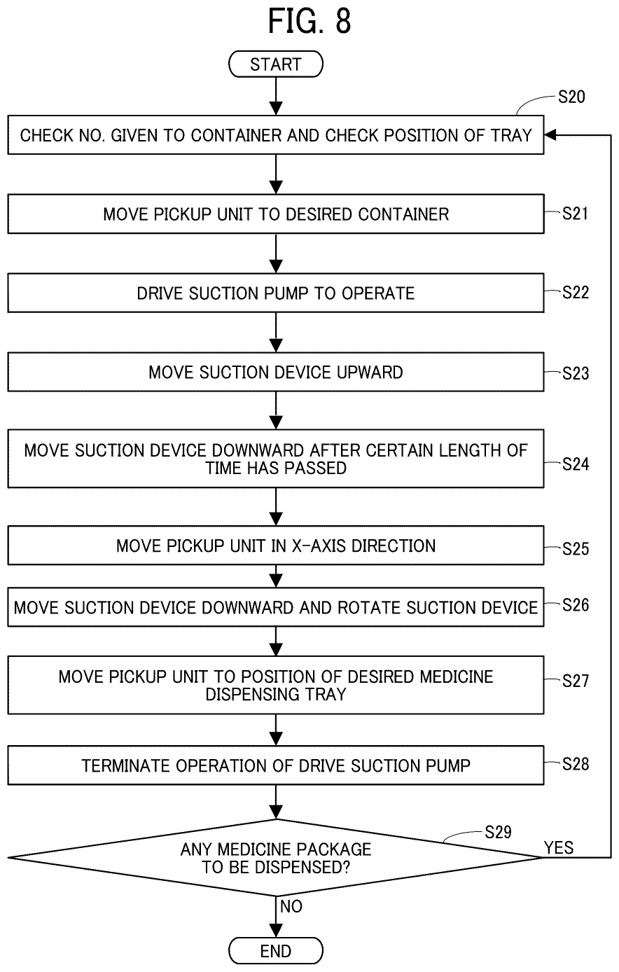

FIG. 8 is a supplemental flowchart of the operation of a pickup device.

In step S20 of FIG. 8, the number (No.) given to desired one of the multiple cartridges 10 that stores the medicine pack 2 to be dispersed is checked, and a desired position of the medicine dispensing tray 30 to which the medicine pack 2 is passed is specified in advance. In other words, the number (No.) given to desired one of the cartridges 10 and the position number of the medicine dispensing tray 30 are specified by the touch panel 151 illustrated in FIG. 3. Subsequently, in step S21, the pickup device 50 is moved to desired one of the multiple cartridges 10 by the conveyance operation of the conveyor 90.

Subsequently, the drive motor 63 is driven to rotate while the suction pump 48 is being driven. As a result, in step S22 and step S23, the suction device 51 moves upward. After certain length of time has passed after the lowermost one of the multiple medicine packs 2 in the storage space 10a of one of one of the multiple cartridges 10 is sucked and held by the pair of suction pads 52, the drive motor 63 is driven to rotate in the reverse direction. Accordingly, in step S24, the suction device 51 moves downward. Subsequently, in step S25, the pickup device 50 is moved in the X-axis direction, and desired one of the multiple medicine packs 2 is completely taken out from one of the multiple cartridges 10. Then, in step S26, the drive motor 63 is further driven to rotate in the reverse direction so as to move the suction device 51 downward. As a result, the suction device 51 is rotated by approximately 90 degrees, and the posture of the medicine pack 2 is changed from an approximately parallel or horizontal posture to an approximately vertical posture.

Subsequently, the pickup device 50 is moved to a desired position of one of the multiple medicine dispensing trays 30 by the conveyor 90. In step S27, the pickup device 50 has moved to the position of desired one of the multiple medicine dispensing trays 30, and then in step S28, the operation of the suction pump 48 is terminated. As a result, the medicine pack 2 is released and separated from the pair of suction pads 52. In so doing, the medicine pack 2 is inserted into the opening 39 of desired one of the subdivision boxes 34 in one of the rooms 33 of the medicine dispensing tray 30.

Subsequently, the bottom end 7 of the inserted medicine pack 2 contacts the inclined portion 37, and simply slides down along the inclined portion 37. In so doing, the medicine pack 2 is stored further inside the subdivision box 34 while the top end of the medicine pack 2 is being tilted backward along a rear side 35. As illustrated in FIG. 5B, the bottom end 7 of the inserted medicine pack 2 contacts a bottom side 38 of the subdivision box 34, and the medicine pack 2 stops moving. Accordingly, a surface of the medicine pack 2 is oriented upward, and medication-related information can be checked as desired.

Then, in step S29, whether there is any medicine pack 2 to be dispensed is checked. When there is no medicine pack 2 to be dispersed, a series of operations is terminated. On the other hand, when it is determined in the step S29 that there is at least one medicine pack 2 to be dispensed, the process is returned to the step S20, and the same series of processes in the above steps are repeated.

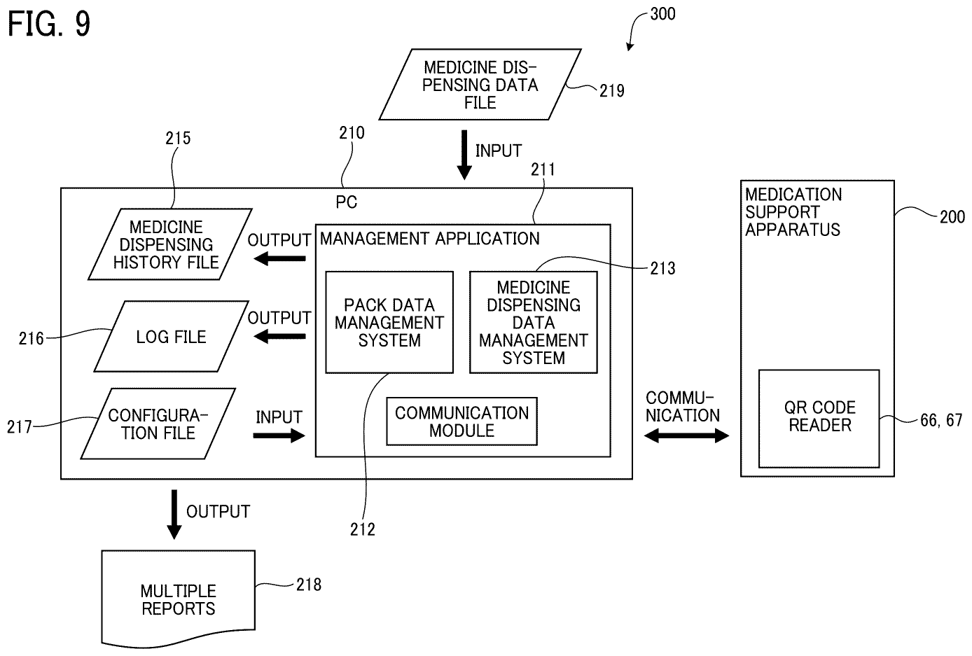

FIG. 9 is a diagram illustrating the control blocks of a medication support system.

As illustrated in FIG. 9, the medication support system 300 is provided with the above-described medication support apparatus 200 and a personal computer (PC) 210 coupled to the medication support apparatus 200. Such coupling enables communication such as data transmission and data reception.

The PC 210 includes five elements. In other words, the PC 210 is provided with, for example, a control device, a processor, an input device, and an output device. The above control device of the PC 210 is provided with a central processing unit (CPU), and executes a program or gives instructions to other devices. The above processor of the PC 210 executes a program or performs computation. The above memory of the PC 210 is provided with, for example, a main memory and an auxiliary memory, and stores data such as programs or texts. The above input device of the PC 210 includes, for example, a mouse, a keyboard, a microphone, and sends data or instructions to the computer. The above output device of the PC 210 includes, for example, a display, a printer, a loudspeaker, and outputs the data output from the computer.

The PC 210 fulfills the function of the medication support apparatus 200, and serves as a host computer that manages and supports the medication support apparatus 200. The PC 210 is connected to a terminal in a pharmacy or the like at which the medicine is supplied, through the network.

The pack data management system 212 provided for the management application 211 manages the medication-related information of the medicine pack. The medicine-dispensing data management system 213 provided for the management application 211 reads and manages the medicine-dispensing data including at least the name of a patient who takes medicines and the times of medication.

For example, the pack data management system 212 and the medicine-dispensing data management system 213 may be implemented as the management application 211 in the PC 210. As illustrated in FIG. 9, a system may include the medication support apparatus 200 and the PC 210 and performs management by communicating with the medication support apparatus 200. The management application 211 reads the medicine-dispensing data file 219 that is externally produced at a pharmacy or the like where medicines are supplied based on the prescription given by a doctor and includes, for example, times at which medicines are dispensed and medicine information for each patient who takes medicines. The management application 211 reads the data output from the configuration file 217 in which, for example, the setting information once applied to the PC 210 is recorded.

For example, a file to be output as log file 216 according to what has been changed or a medicine dispensing history file 215 in which medicine dispensing history is tracked is the data to be output from the management application 211 as necessary. Further, the multiple reports 218 are output as necessary from the PC 210 through the management application 211. The functions such as of the management application 211 of the PC 210 may be arranged in the medication support apparatus 200.

In the medicine-dispensing data management system 213, the results of medicine dispensing or the change history are stored as record. By so doing, the actual medicine dispensing results are stored as traceability information. This enables a review when a problem occurs.

The pack data management system 212 performs management based on the medication-related information of the medicine pack including at least the name of a patient who takes medicines and the times of medication. However, no limitation is indicated thereby, and a supplementary explanation of the operation of, for example, the medication support apparatus 200 described as above is given below.

The pickup device 50 that is an example of a carriage is provided with an upper quick response (QR) code reader 66 and a lower QR code reader 67 in the medication support apparatus 200. The pickup device 50 moves close to one of the multiple cartridges 10 that stores a medicine pack such as a medicine package and bound medicine packages, and the QR CODE that indicates the medication-related information can be read by the upper reader unit of the upper QR code reader 66.

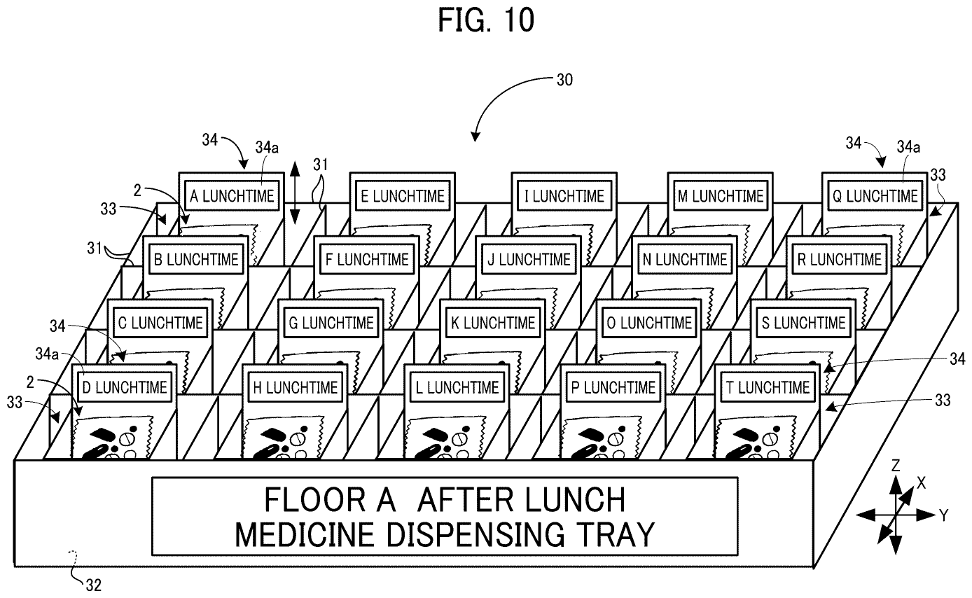

FIG. 10 is an external perspective view of the medicine dispensing tray 30.

As illustrated in FIG. 10, each one of the multiple medicine dispensing trays 30 has a plurality of partition walls 31 that is an example of a plurality of dividers, partitions, or partition walls used to arrange prescribed packs, and is partitioned into twenty rooms 33 by four upright partition walls 31. The twenty rooms 33 that are formed in each one of the multiple medicine dispensing trays 30 can be expressed as the components of a matrix including five columns in the X-axis direction or the line feed direction and four rows in the Y-axis direction or the character feed direction. As a result, each one of the twenty rooms 33 of the medicine dispensing trays 30 can be uniquely positioned based on the components and addresses of the matrix of five columns and four rows.

Furthermore, each one of the multiple medicine dispensing trays 30 has a bottom wall 32 on which the multiple medicine packs 2 are placed. As described above, in the medicine dispensing tray 30, specific one of the multiple packs is placed in specific one of the multiple rooms 33 in a reliable manner by the multiple partition walls 31, which are four partition walls in the present embodiment, and a bottom wall 32 in common, so as not to be mixed up with packs in different one of the multiple rooms 33 or drop off from the bottom wall 32.

The medicine dispensing tray 30 illustrated in FIG. 10 includes the twenty rooms 33 that serve as a group of medicine dispensers as a whole and subdivision boxes 34 that serve as individual medicine dispensers detachable from the rooms 33. The medicine dispensing tray 30 serves as a collective medicine dispenser. The medicine dispensing tray 30 is provided with the partition walls 31 and is divided into twenty rooms 33 such that the medicine packs 2 can be stored separately.

The subdivision box 34 is detachable from the rooms 33 in which the medicine packs are arranged. For example, as illustrated in FIG. 10, the multiple subdivision boxes 34 hold the multiple medicine packs 2 filled with the medicines 3 to be taken by twenty residents A to T after lunch in a day-care center or nursing home. In other words, one of the multiple medicine dispensing trays 30 illustrated in FIG. 10 is used to arrange prescribed packs in a predetermined or specific one of the multiple rooms 33 partitioned by dividers through the multiple subdivision boxes 34.

When a personal care attendant who is a staff in a day-care center or nursing home and helps a person who takes medicines to takes the medicines sorts medicines such as the medicines in a medicine pack to be taken on that day in advance using the medication support apparatus 200 as above, medicine packs are sorted into the subdivision boxes 34 of the rooms 33 of the medicine dispensing tray 30 as illustrated in FIG. 10 in advance for each occupant. By so doing, only the medicine packs to be taken can be brought to a site such as a canteen or the private room of an occupant where medication takes place. This can reduce the chances of medication error where an occupant takes medicine at a wrong time of medication. The medicine dispensing tray 30 may be prepared for each time of medication such as a time in the morning, a time at lunchtime, a time in the evening, and a time before going to the bed, or medicine packs may be sorted and managed before each time of medication using a single medicine dispensing tray or multiple medicine dispensing trays.

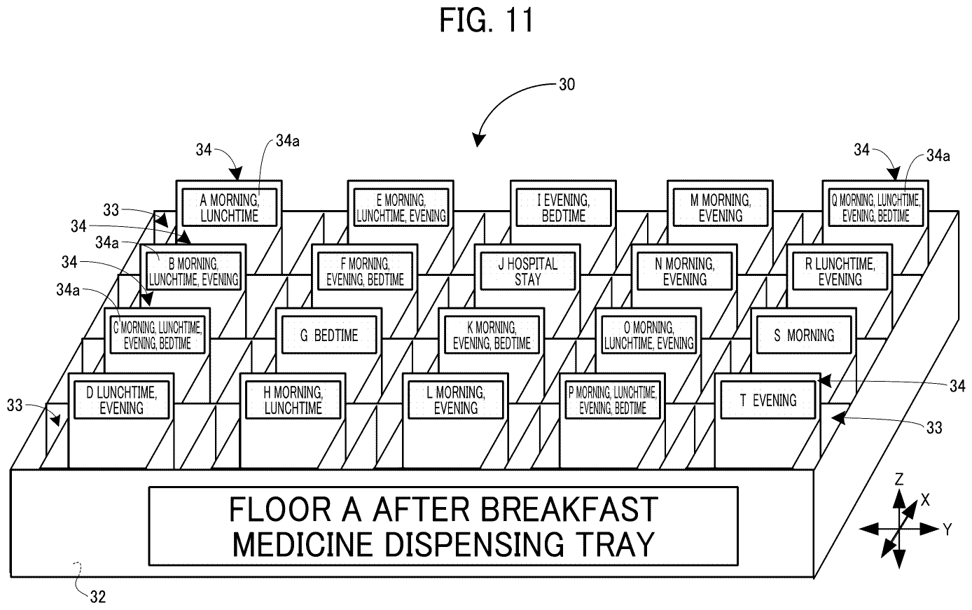

As in the medicine dispensing tray 30 illustrated in FIG. 10, a display label 34a that displays the name of an occupant is attached to an upper portion of the subdivision box 34 of each of the rooms 33, and the position of the subdivision box 34 in the rooms 33 is determined for each occupant. For example, as illustrated in FIG. 11, the times of medication vary for each occupant. For example, occupant A takes medicines in the morning and at lunchtime, but occupant B takes medicines in the morning, at lunchtime, and in the evening. Occupant C takes medicines in the morning, at lunchtime, in the evening, and before going to the bed. As the positions of occupants in the medicine dispensing tray 30 are determined and displayed in advance, wrong administration of medicines while a person who takes medicines is being assisted to take the medicines can be prevented, and the chances of medication error can be reduced.

When the medicine dispensing tray 30 is prepared at each times of medication, the displays of occupants D, G, I, J, R, and T may be erased from the medicine dispensing tray 30 after the breakfast as illustrated in FIG. 11 or the subdivision boxes 34 may be removed to indicate that it is not necessary to prepare any medicine pack.

When the medicine dispensing tray 30 is to be reused, as illustrated in FIG. 10 and FIG. 11, times of medication are written or drawn beforehand near the name of occupants to indicate medicine packs to be prepared.

In each one of the multiple rooms 33 of the multiple medicine dispensing trays 30, a setting position or inserting position is determined for each patient who takes medicines depending on the type of medicines in the pack to be taken. In other words, the multiple rooms 33 in one of the multiple medicine dispensing trays 30 may be allocated to patients who take medicines at the same time. In a case where a specific patient who is to take medicines does not take a medicine at a specific time of medication, the arrangement can be made such that a pack is not given to such a specific patient at the specific time of medication.

However, no limitation is intended by the medicine dispensing trays 30 described above. Each one of the multiple rooms 33 of one of the medicine dispensing trays 30 may be allocated for each time of medication of each patient who takes medicines. More specifically, in such an alternative embodiment of the present disclosure, the multiple rooms 33 may be allocated to each of the patients who take medicines in view of the time of medication of each one of the multiple medicine packs 2 to be taken in the morning, in the daytime, in the evening, or before going to bed. The medicine dispensing trays 30 of such cases may be managed on a room-by-room basis or a floor-by-floor basis in day-care centers or nursing homes where patients who take medicines reside, and the multiple medicine packs 2 for that day or a couple of days may be dispersed in advance to the multiple medicine dispensing trays 30.

According to the above alternative embodiment, allocating the rooms 33 based on each time of medication such as a time in the morning, a time in the daytime, a time in the evening, and a time before going to bed can prevent the time of medication from being mistaken by patients who take medicines. The configuration or structure of the multiple medicine dispensing trays 30 is not limited to the embodiments of the present disclosure described above, and various kinds of combinations are theoretically possible between patients who take medicines and the timing at which the medicines are to be taken. However, such combinations may go beyond the scope of the present disclosure, the description of such combinations is omitted.

A configuration and operation of a remaining-amount sensor that detects the remaining amount of the multiple medicine packs 2 stored in the storage space 10a of the cartridge 10 in the medication support apparatus 200 are described below.

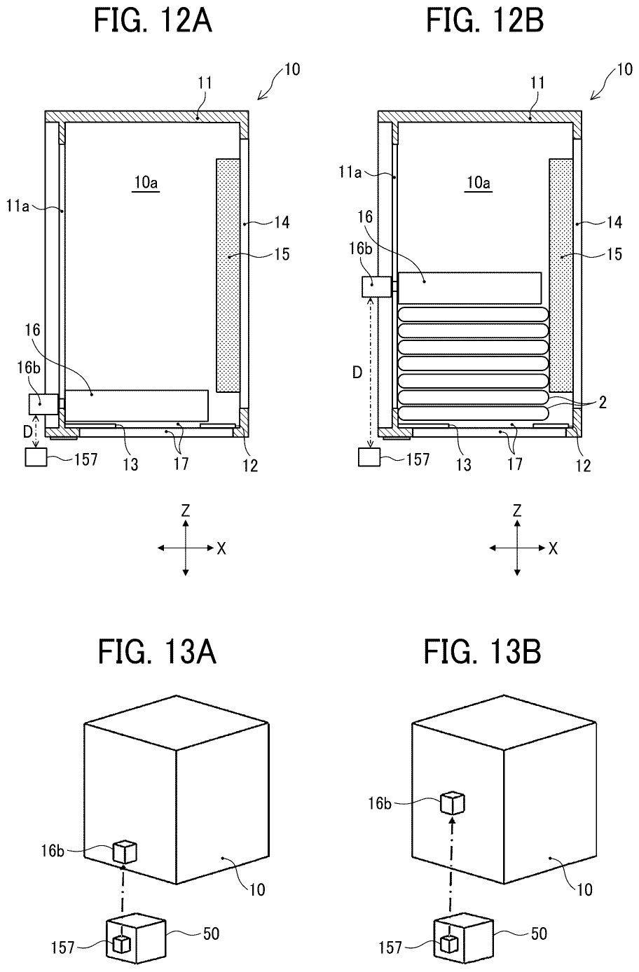

FIG. 12A and FIG. 12B are diagrams each of which illustrates a remaining-amount sensor 157.

As illustrated in FIG. 12A, the feeler 16b of the movable board 16, which is an example of a mover, is at an end position, i.e., the second position, that corresponds to the second remaining amount, which is zero in the present embodiment. As illustrated in FIG. 12B, the feeler 16b of the movable board 16 is at a near end position, i.e., the first position, that corresponds to the first remaining amount, which is a few in the present embodiment.

The remaining-amount sensor 157 that is an example of a sensor and detects the position of the feeler 16b attached to the movable board 16 of the cartridge 10 is attached to the cartridge tray 27 to which the cartridges 10 are placed. The remaining-amount sensor 157 is, for example, a range sensor such as a time-of-flight (TOF) sensor, and is arranged on the extension of the route of travel of the feeler 16b of the movable board 16 that moves or slide according to a reduction in the remaining amount of the medicine packs 2 inside the storage space 10a of the cartridge 10.

When the medicine pack 2 is picked up from the storage space 10a of the cartridge 10, the remaining amount of the medicine pack 2 in the storage space 10a decreases, and the length of the medicine pack 2 in the Z-axis direction, i.e., the aligned direction of the medicine packs, changes accordingly. In other words, the overall thickness of the medicine packs 2 that are stacked on top of each other in layers changes. Accordingly, the movable board 16 that is placed upon the uppermost one of the medicine packs 2 that are stacked on top of each other in layers inside the storage space 10a of the cartridge 10 moves downward by its own weight in the Z-axis direction inside the storage space 10a. As a result, the feeler 16b of the movable board 16 moves downward in the Z-axis direction toward the remaining-amount sensor 157.

As described above, the remaining-amount sensor 157 measures the distances D to the feeler 16b that moves according to a reduction in the remaining amount of the medicine packs 2 inside the storage space 10a, and outputs the distances D as the measurement results to the controller 150. As the distances D that are measured by the remaining-amount sensor 157 correspond to the positions of the feeler 16b, the controller 150 can figure out the position of the feeler 16b based on the distance D measured by the remaining-amount sensor 157.

FIG. 13A and FIG. 13B are diagrams each of which illustrates the remaining-amount sensor 157.

The feeler 16b of the movable board 16 is at an end position in FIG. 13A, and the feeler 16b of the movable board 16 is at a near end position in FIG. 13B.

The remaining-amount sensor 157 may be fixed to, for example, the cartridge tray 27 of the medication support apparatus 200. However, in such a configuration, the remaining-amount sensor 157 is to be provided for each one of the cartridges 10, and the remaining-amount sensor 157 has to be provided as many as the number of the cartridges 10.

In order to avoid such a situation, in the present embodiment, the pickup device 50 that picks up the medicine packs 2 from the storage space 10a of one of the multiple cartridges 10 is provided with the remaining-amount sensor 157. The pickup device 50 is moved to pack-pickup positions under the first cartridges 10 by the conveyor 90, and picks up the medicine packs 2 from each one of the cartridges 10. In view of such circumstances, the pickup device 50 is provided with the remaining-amount sensor 157 By so doing, the pickup device 50 is moved to pack-pickup positions under the first cartridges 10 by the conveyor 90, and the distances D to the feelers 16b of multiple cartridges 10 can be measured using a single remaining-amount sensor 157.

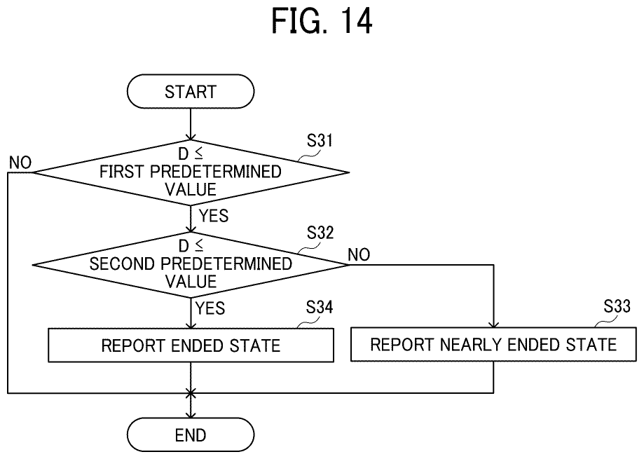

FIG. 14 is a flowchart of remaining-amount determining processes when a remaining amount is detected.

When the position of the feeler 16b is determined based on the distance D measured by the remaining-amount sensor 157, in step S30, the controller 150 starts the remaining-amount determining processes are performed. Then, in step S31, the controller 150 determines whether the obtained distance D is equal to or smaller than a first predetermined value that corresponds to a near end position (first position) as illustrated in FIG. 12B.

When it is determined that the obtained distance D is not equal to or smaller than the first predetermined value (NO in step S31), the remaining-amount determining processes are terminated. When a nearly ended state or ended state, as will be described later in detail, is continuing at that moment in time, such a state is to be released. By contrast, when it is determined that the obtained distance D is equal to or smaller than the first predetermined value (YES in step S31), in step S32, the controller 150 determines whether the obtained distance D is equal to or smaller than a second predetermined value, which is smaller than the first predetermined value.

When it is determined that the obtained distance D is not equal to or smaller than the second predetermined value (NO in step S32), the controller 150 determines that the feeler 16b has moved to the near end position, i.e., the first position, that corresponds to a near-end remaining amount, i.e., the first remaining amount, and in step S33, controls the notifier 154 to notify a user that the cartridge 10 has reached the nearly ended state. By contrast, when it is determined that the obtained distance D is equal to or shorter than the second predetermined value (YES in step S32), the controller 150 determines that the feeler 16b has moved to the end position, i.e., the second position, that corresponds to an end remaining amount, i.e., the second remaining amount, and in step S34, controls the notifier 154 to notify a user that the cartridge 10 has reached the ended state.

The thickness T1 of a single medicine pack 2 stored in the storage space 10a of the cartridge 10 varies depending on, for example, differences in the time of medication of that medicine pack 2, differences in the amount of medicine to be taken, and differences in the type of medicine. Accordingly, the first predetermined value, which corresponds to the near end position and is used to determine that the cartridge 10 is in a nearly ended state, varies depending on differences in the thickness T1 of a single medicine pack 2 stored in the storage space 10a of the cartridge 10. In order to handle such a situation, the first predetermined value may be changed as necessary by, for example, the input operation made on the touch panel 151.

In the present embodiment, two predetermined values including the first predetermined value and the second predetermined value are used to determine whether the cartridge 10 is in a nearly ended state or ended state. However, no limitation is indicated thereby, and only one predetermined value may be used. By way of example, when the remaining amount of the medicine packs 2 inside the storage space 10a of the cartridge 10 is to be determined in multiple levels, three or more predetermined values may be used. For example, a third predetermined value may be added to determine whether the medicine packs 2 inside the storage space 10a of the cartridge 10 have reached capacity.

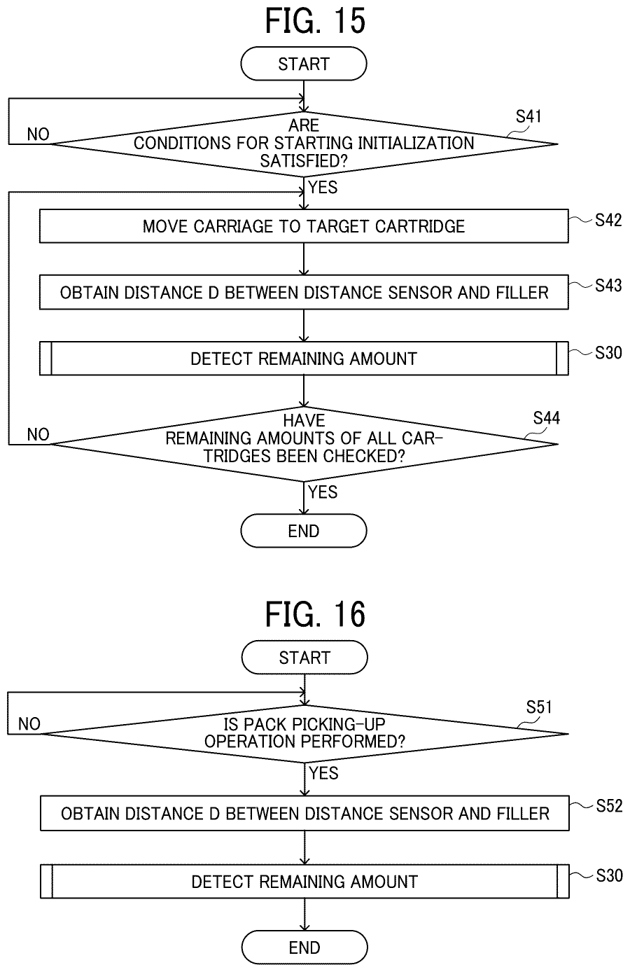

FIG. 15 is a flowchart of a remaining-amount detecting operation at the time of initialization.

When predetermined conditions for starting initialization are satisfied (YES in step S41), the controller 150 starts the remaining-amount detecting operation at the time of initialization. Such predetermined conditions for starting initialization include, for example, a condition that a user has changed the power of the medication support apparatus 200 from OFF to ON and a condition that the cartridge tray 27 is drawn out from the medication support apparatus 200 through the drawer 21. It is desired that the predetermined conditions for starting initialization be, for example, conditions that allow a user to supply the medicine packs 2 to that cartridge 10.

When the remaining-amount detecting operation at the time of initialization is started, in step S42, the controller 150 controls the conveyor 90 to move the pickup device 50, which is an example of a carriage, to a pack-pickup position under the first cartridge 10. In step S43, the controller 150 obtains the distance D to the feeler 16b of that first cartridge 10 using the remaining-amount sensor 157 attached to the pickup device 50. Subsequently, in steps S31 to S34, the controller 150 executes the remaining-amount determining processes in step S30 as depicted in FIG. 14.

When the remaining-amount determining processes S30 are completed, in step S42, the controller 150 controls the conveyor 90 to move the pickup device 50, which is an example of a carriage, to a pack-pickup position under the next cartridge 10. In step S43, the controller 150 obtains the distance D between the next cartridge 10 and feeler 16b, and then the controller 150 executes the remaining-amount determining processes S30 in steps S31 to S34. When the above processes are repeated and the remaining-amount determining processes S30 have been completed for all the cartridges 10 (YES in step S44), the remaining-amount detecting operation is terminated.

In the remaining-amount detecting operation in step S40 at the time of initialization according to the present embodiment, a remaining-amount detecting operation is performed for all the cartridges 10 placed in the medication support apparatus 200. However, no limitation is indicated thereby, and a remaining-amount detecting operation may be performed for some of the cartridges 10 placed in the medication support apparatus 200. For example, a remaining-amount detecting operation may be performed for the cartridges 10 selected by a user. In such cases, the length of time required to perform a remaining-amount detecting operation in step S40 at the time of initialization can be reduced.

FIG. 16 is a flowchart of a remaining-amount detecting operation S50 at the time of pack picking-up operation where a medicine pack is picked up from the cartridge 10.

The controller 150 controls the conveyor 90 moves the pickup device 50, which is an example of a carriage, to a pack-pickup position under the cartridge 10. When some of the medicine packs 2 are picked up from that cartridge (YES in step S51), in step S52, the controller 150 obtains the distance D to the feeler 16b of that cartridge 10 using the remaining-amount sensor 157 attached to the pickup device 50. Subsequently, in steps S31 to S34, the controller 150 executes the remaining-amount determining processes in step S30 as depicted in FIG. 14.

As described above, a remaining-amount detecting operation is performed for the cartridge 10 together with the picking-up operation for the medicine packs 2 from the cartridge 10 to omit the movement of the pickup device 50, which is an example of a carriage, for the remaining-amount detecting operation.

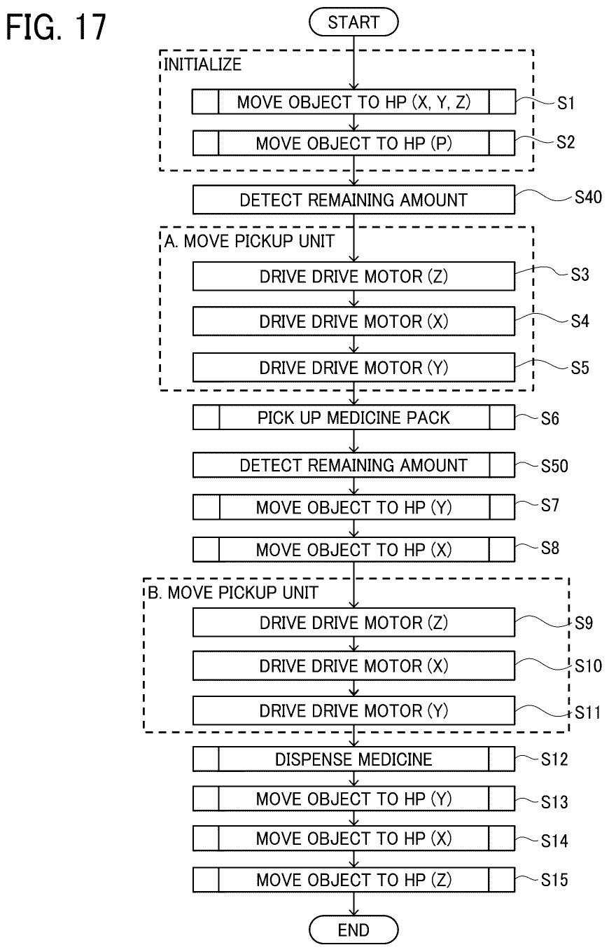

FIG. 17 is a flowchart of basic operations of the medication support apparatus 200, including a remaining-amount detecting operation S40 at the time of initialization as depicted in FIG. 15 and a remaining-amount detecting operation S50 at the time of pack picking-up operation as depicted in FIG. 16.

In other words, in the flowchart of FIG. 17, a remaining-amount detecting operation in step S40 at the time of initialization and a remaining-amount detecting operation in step S50 at the time of pack picking-up operation are added to the flowchart depicted in FIG. 6.

As depicted in FIG. 17, the remaining-amount detecting operation in step S40 at the time of initialization as depicted in FIG. 15 is performed after the medication support apparatus 200 is initialized in steps S1 and S2. The remaining-amount detecting operation S50 in the picking-up operation for the medicine packs 2 from the cartridge 10, which is depicted in FIG. 16, is performed after the medicine pack 2 is picked up from the cartridge 10 in step S6.

### First Modification

The first modification of the above-described embodiment is described below. In the embodiments of the present disclosure as described above, the sensor is configured by a single remaining-amount sensor 157 composed of a range sensor. By contrast, in the first modification, the sensor is implemented by a relatively simple transmissive optical sensor. Overlapping descriptions with the description of the embodiments as described above are omitted where appropriate.

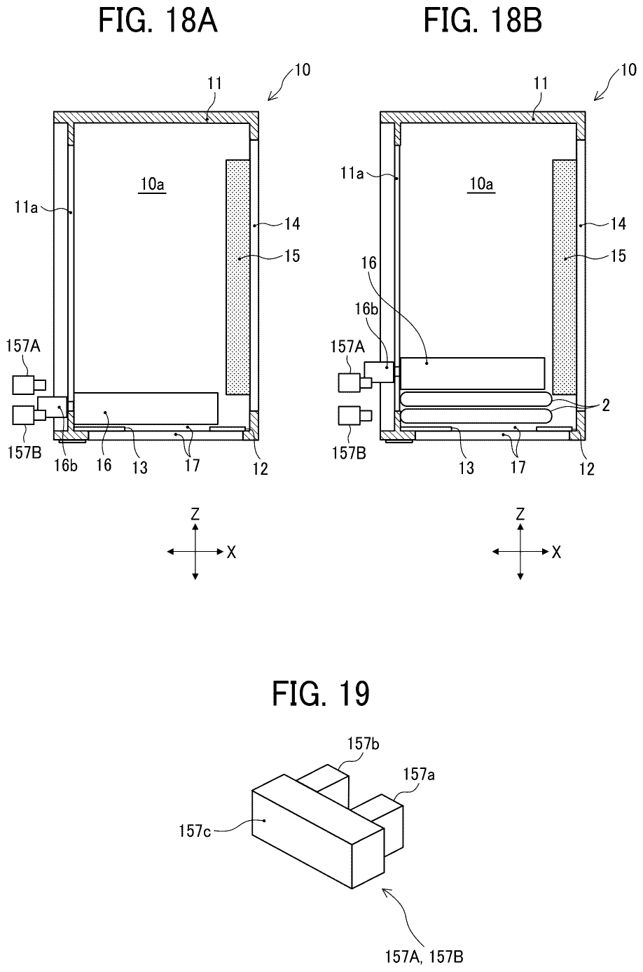

FIG. 18A and FIG. 18B are diagrams each of which illustrates a configuration and operation of a sensor implemented by a transmissive optical sensor, according to the first modification.

FIG. 19 is a schematic perspective view of the transmissive optical sensor.

The feeler 16b of the movable board 16 is at an end position in FIG. 18A, and the feeler 16b of the movable board 16 is at a near end position in FIG. 18B.

The sensor according to the first modification includes a near-end sensor 157A, i.e., a transmissive optical sensor, arranged such that the feeler 16b at a near end position blocks the optical path, and an end sensor 157B, i.e., a transmissive optical sensor, arranged such that the feeler 16b at an end position blocks the optical path.

Every time the medicine pack 2 is picked up from the cartridge 10, the feeler 16b and the movable board 16 move downward in the Z-axis direction. When the feeler 16b reaches the near end position, the optical path between the light emitter 157a and the light receiver 157b of the near-end sensor 157A is blocked, and the radiation intensity of light received by the light receiver 157b decreases. The controller 150 that receives the output from the near-end sensor 157A can determine that the cartridge 10 has entered the nearly ended state by detecting the decrease in the radiation intensity of received light, and notifies a user of such a state.

When the feeler 16b reaches the end position, the optical path between the light emitter 157a and the light receiver 157b of the end sensor 157B is blocked, and the radiation intensity of light received by the light receiver 157b of the end sensor 157B decreases. The controller 150 that receives the output from the end sensor 157B can determine that the cartridge 10 has entered the ended state by detecting the decrease in the radiation intensity of received light, and notifies a user of such a state.

As described above, the thickness T1 of a single medicine pack 2 stored in the storage space 10a of the cartridge 10 varies depending on, for example, differences in the time of medication of that medicine pack 2, differences in the amount of medicine to be taken, and differences in the type of medicine. Accordingly, the near end position that is used to determine that the cartridge 10 is in a nearly ended state, varies depending on differences in the thickness T1 of a single medicine pack 2 stored in the storage space 10a of the cartridge 10. For this reason, it is desired that the near end position changeable. For example, a plurality of slot for fixation may be arranged on an attaching bracket 157c of the near-end sensor 157A. By so doing, the installation position of the near-end sensor 157A in the Z-axis direction is made changeable, or the near-end sensor 157A is made movable in the Z-axis direction by a drive motor. A plurality of near-end sensors 157A may be provided.

### Second Modification

The second modification of the above-described embodiment is described below.

In the second modification, a fixed single sensor performs a remaining-amount detecting operation for the multiple cartridges 10. In a similar manner to the first modification as described above, the sensor according to the second modification is configured by the near-end sensor 157A and the end sensor 157B each of which is implemented by a transmissive optical sensor. Overlapping descriptions with the description of the first modification and the embodiments as described above are omitted where appropriate.

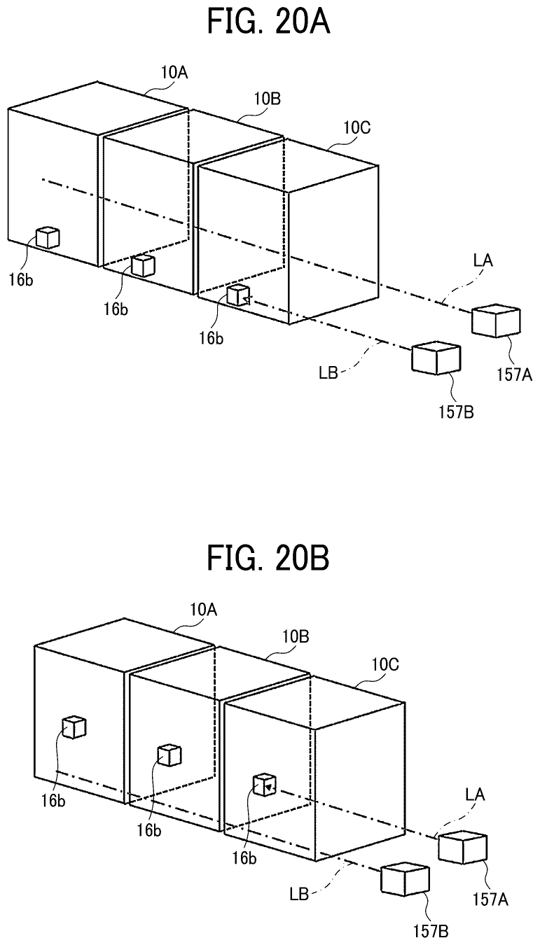

FIG. 20A and FIG. 20B are diagrams each of which illustrates a configuration and operation of a sensor implemented by a transmissive optical sensor, according to the second modification.

The feeler 16b of the movable board 16 is at an end position in FIG. 20A, and the feeler 16b of the movable board 16 is at a near end position in FIG. 20B.

In the second modification, the near-end sensor 157A and the end sensor 157B are arranged for the three cartridges 10A to 10C such that all the feelers 16b of the three cartridges 10A to 10C intersect with both the optical paths LA and LB of the near-end sensor 157A and the end sensor 157B. In such a configuration, it is determined that the cartridge 10 is in a nearly ended state when at least one of the feelers 16b of the three cartridges 10A to 10C is located at a position where an optical path LA of the near-end sensor 157A is blocked. In a similar manner to the above, it is determined that the cartridge 10 is in an ended state when at least one of the feelers 16b of the three cartridges 10A to 10C is located at a position where an optical path LB of the end sensor 157B is blocked.

According to the second modification, the number of sensors can be reduced to simplify the configuration or structure of the sensors. When one of the cartridges 10 has entered a nearly ended state, typically, a user supplies the medicine packs 2 to that cartridge 10 and also supplies the medicine packs 2 to other ones of the cartridge 10. The second modification is suited for such an operation.

In the second modification, the number of the cartridges 10 whose remaining amounts are detected by a single sensor such as a single near-end sensor 157A and a single end sensor 157B is three. However, no limitation is indicated thereby, and the number of the cartridges 10 whose remaining amounts are detected by a single sensor may be two or four or more.

### Third Modification

Yet another modification of the above-described embodiment is described below.

In the third modification, the one-pack thickness calculating processes are performed, and the amount of movement or displacement of the feeler 16b per each measurement unit in a one-time picking-up operation for the medicine packs 2, which is the thickness T1 of a single medicine pack 2, is obtained. Then, the remaining amount of the medicine packs 2 inside the storage space 10a of the cartridge 10 can be determined with a high degree of precision, based on the obtained amounts of movement per each measurement unit, which is the thickness of a single medicine pack 2. In a similar manner to the embodiments of the present disclosure as described above, the sensor according to the third modification is configured by the remaining-amount sensor 157 including a range sensor. Overlapping descriptions with the description of the embodiments as described above are omitted where appropriate.

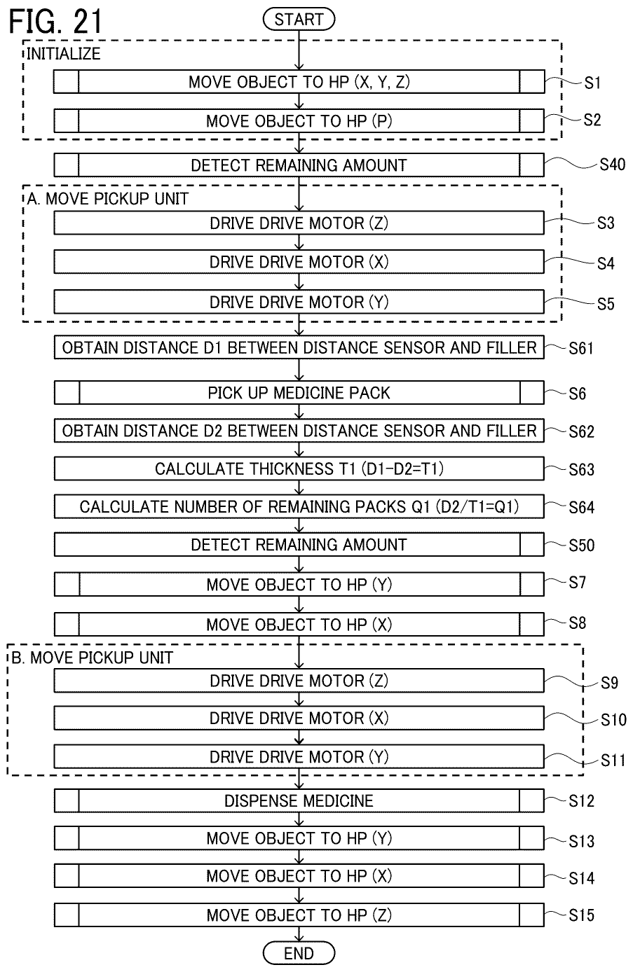

FIG. 21 is a flowchart of basic operations of the medication support apparatus 200, including one-pack thickness calculating processes, according to the third modification.

In other words, in the flowchart of FIG. 21, one-pack thickness calculating processes are added to the flowchart depicted in FIG. 17.

In the one-pack thickness calculating processes according to the third modification, before the picking-up operation S6 for the medicine packs 2 by the pickup device 50 from the cartridge 10, in step S61, distance D1 to the feeler 16b is measured using the remaining-amount sensor 157 that serves as a range sensor and the controller 150 obtains the distance D1 as the measurement result. Subsequently, in step S6, the pickup device 50 picks up one of the medicine packs 2 from the cartridge 10. Before the pickup device 50, which is an example of a carriage, starts moving away from a pack-pickup position under the cartridge 10, in step S62, a distance D2 to the feeler 16b is measured again using the remaining-amount sensor 157 that serves as a range sensor, and the controller 150 obtains the distance D2 as the measurement result.

The difference between the distance D1 that indicates the position of the feeler 16b and correspond to the remaining amount of medicine packs before a single medicine pack 2 is taken out and the distance D2 that indicates the position of the feeler 16b and correspond to the remaining amount of medicine packs after that medicine pack 2 has been taken out indicates the distance T1 that corresponds to the thickness T1 of the medicine pack 2, which is the amount of movement of the feeler 16b per each measurement unit. In step S63, the controller 150 calculates the difference between the distance D1 and the distance D2 to obtain the thickness T1 of a single medicine pack 2.

As described above, the thickness T1 of a single medicine pack 2 stored in the storage space 10a of the cartridge 10 can be obtained, and the number of the medicine packs 2 remaining in the cartridges 10 can be obtained with a high degree of precision. Accordingly, a user can be notified of an accurate number of the medicine packs 2 remaining in the cartridges 10.

More specifically, in step S64, the number of remaining packs Q1 of the cartridges 10 can be obtained accurately by dividing the distance D2 calculated in step S62 after the picking-up operation S6 for the medicine packs 2 by the thickness T1 of a single medicine pack 2, which is calculated in step S63.

### Fourth Modification

Yet another modification of the above-described embodiment is described below.

Also in the fourth modification, in a similar manner to the third modification as described above, the one-pack thickness calculating processes in which the amount of movement or displacement of the feeler 16b per each measurement unit in a one-time picking-up operation for the medicine packs 2, which is the thickness T1 of a single medicine pack 2, is obtained are performed. However, the fourth modification is different from the above-described third modification in that whether to retry the pack picking-up operation is determined using the obtained amounts of movement per each measurement unit, which is the thickness of a single medicine pack 2. Overlapping descriptions with the description of the third modification as described above are omitted where appropriate.

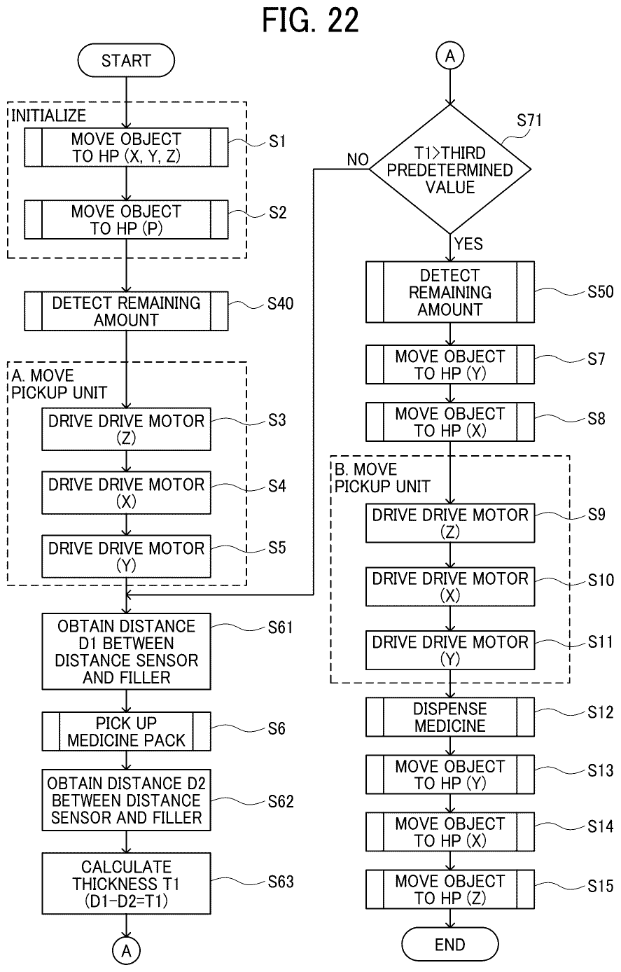

FIG. 22 is a flowchart of basic operations of the medication support apparatus 200, including a pickup retrying operation, according to the fourth modification.

In other words, step S71 to determine whether to retry the pack picking-up operation is added to the flowchart of FIG. 22 in place of step S64 depicted in the flowchart of FIG. 21.

In the fourth modification, the controller 150 obtains the thickness T1 of a single medicine pack 2 in one-pack thickness calculating processes S61 to S63, and then in step S71, determines whether the obtained value of the thickness T1 is greater than the third predetermined value. The third predetermined value is designed to have a value less than at least the value of the thickness T1 of a single medicine pack 2. In view of the variations in the value of the thickness T1, it is desired that the third predetermined value have a value about one-third of the value of the thickness T1.

When the obtained value of the thickness T1 is greater than the third predetermined value (YES in step S71), it is determined that some of the medicine packs 2 are taken out in the previous pack picking-up operation S6, and the process is advanced without interruption. By contrast, when it is determined that the obtained value of the thickness T1 is equal to or smaller than the third predetermined value (NO in step S71), it is determined that none of the medicine packs 2 has been taken out in the previous pack picking-up operation S6. In such a case, the controller 150 returns the process to step S61 again, and retries the pack picking-up operation S6. After such a retry, the process continues on an as-is basis when it is later determined that the obtained value of the thickness T1 is greater than the third predetermined value (YES in step S71).

A retrying operation may be performed after fine adjustments are performed on a position in the X-axis direction, the Y-axis direction, and the Z-axis direction in a range where the remaining-amount sensor 157 that serves as a range sensor can read a normal value.

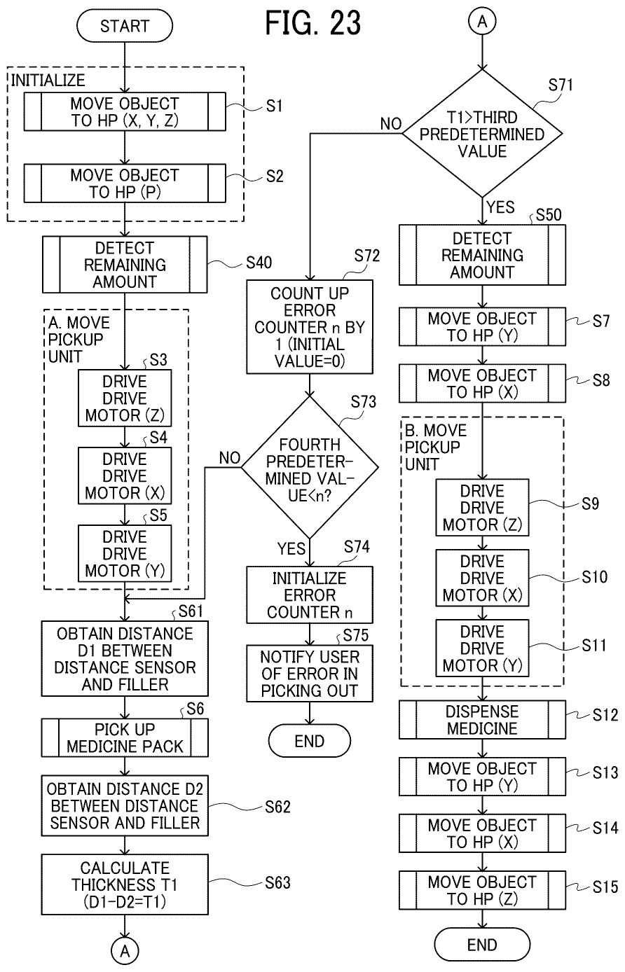

FIG. 23 is a flowchart of the operations of FIG. 22 to which picking-out error processes are added.

When the medicine packs 2 could not successfully be picked up in the pack picking-up operation S6, a user is notified of a picking-out error in the picking-out error processes.

The controller 150 obtains the thickness T1 of a single medicine pack 2 in one-pack thickness calculating processes S61 to S63. When it is determined that the obtained value of the thickness T1 is equal to or smaller than the third predetermined value (NO in step S71), firstly, in step S72, the controller 150 counts up an error counter n by 1, where the initial value of n is 0, and then, in step S73, the controller 150 compares the fourth predetermined value with the error counter n.

When the error counter n is equal to or smaller than the fourth predetermined value in such a comparison (NO in step S73), the controller 150 returns the process to step S61 again, and retries the pack picking-up operation S6. When it is determined in later step S71 that the obtained value of the thickness T1 is greater than the third predetermined value after the retry (YES in step S71), the process continues on an as-is basis.

By contrast, when it is kept determined that the obtained value of the thickness T1 is equal to or smaller than the third predetermined value after the retry (NO in step S71) and the error counter n exceeds the fourth predetermined value (step S72, YES in step S73), in step S74, the error counter n is initialized. Then, in step S75, a user is notified of a picking-out error, and this series of processes is terminated.

The fourth predetermined value is satisfactory as long as it is equal to or greater than one. However, as the value increases, it is more likely that downtime occurs. For this reason, it is desired that the fourth predetermined value be about three.

### Fifth Modification

Yet another modification of the above-described embodiment is described below. Also in the fifth modification, in a similar manner to the third modification as described above, the one-pack thickness calculating processes in which the amount of movement or displacement of the feeler 16b per each measurement unit in a one-time picking-up operation for the medicine packs 2 are performed. In other words, the thickness T1 of a single medicine pack 2 is obtained in the one-pack thickness calculating processes. However, the fifth modification is different from the above-described third modification in that an error in pickup due to multiple feeding is detected using the obtained amounts of movement per each measurement unit, which is the thickness of a single medicine pack 2. Overlapping descriptions with the description of the third modification as described above are omitted where appropriate.

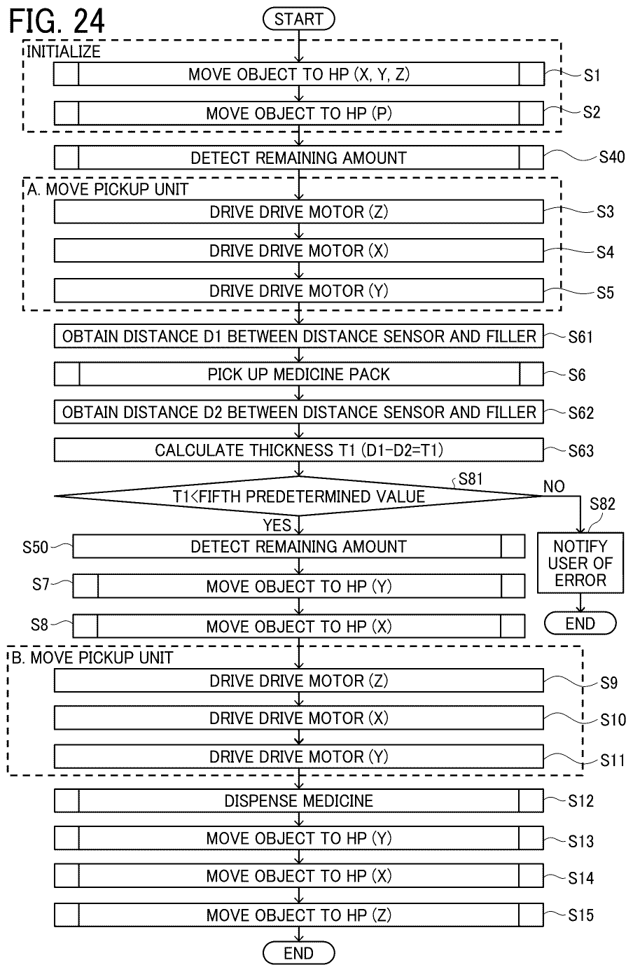

FIG. 24 is a flowchart of basic operations of the medication support apparatus 200, including the operations to deal with an error in pickup due to multiple feeding, according to the fifth modification.

In other words, step S81 to detect an error in pickup due to multiple feeding is added to the flowchart of FIG. 24 in place of step S64 depicted in the flowchart of FIG. 21.

In the fifth modification, the controller 150 obtains the thickness T1 of a single medicine pack 2 in one-pack thickness calculating processes S61 to S63, and then in step S81, determines whether the obtained value of the thickness T1 is smaller than a fifth predetermined value. Ideally, the fifth predetermined value is about the twice as much as the thickness T1. However, the thickness T1 varies to some degree due to, for example, the posture or attitude of the tablet contained in the medicine pack 2. For this reason, it is desired that the fifth predetermined value have a value about 1.5 times the value of the thickness T1. As the thickness T1 varies depending on, for example, the type of medicine, the fifth predetermined value is made variable.

When the obtained value of the thickness T1 is smaller than the fifth predetermined value (YES in the step S81), it is determined that the medicine packs 2 are not fed in pair in the previous pack picking-up operation S6, and the process is advanced without interruption. Note that when the medicine packs 2 are fed in pair and multiple feeding takes place erroneously, a greater number of medicine packs than is necessary are erroneously picked up.

By contrast, when it is determined that the obtained value of the thickness T1 is equal to or greater than the fifth predetermined value (NO in step S81), it is determined that some of the medicine packs 2 are fed in pair in the previous pack picking-up operation S6. Then, in step S82, a user is notified of an error in pickup due to multiple feeding, and this series of processes is terminated.

The embodiments described above are given as an example, and advantageous effects are achieved for each of the following modes given below.

### First Aspect

In a medicine-pack housing such as the cartridge 10, medicine packs are picked up by the pickup device 50 from a container such as the storage space 10a inside the casing 11 in which a plurality of medicine packs such as the medicine packs 2 are aligned in line and stored. The medicine-pack housing includes a mover such as the feeler 16b to move in synchronization with a change in a length of the plurality of medicine packs in an aligned direction of the plurality of medicine packs, which is the overall thickness of the medicine packs 2 in the Z-axis direction, due to a reduction in a remaining amount of the plurality of medicine packs inside the container, a sensor such as the remaining-amount sensor 157, the near-end sensor 157A, and the end sensor 157B to detect a position of the mover, and a remaining-amount determination unit such as the remaining-amount calculator 159 the controller 150 to determine the remaining amount of the plurality of medicine packs inside the container based on a detection result of the sensor.

In a medicine-pack housing according to a comparative example, the container has to be placed onto a measuring instrument in order for the measuring instrument to measure the weight of a container that stores medicine packs. Accordingly, at least, the container placed in the medicine-pack housing has to be lifted and brought down onto the measuring instrument, and it takes a long time to determine the remaining amount of the medicine packs inside the container. In particular, there are some cases in which the measuring instrument cannot perform measurement accurately until the movement or shake that is caused as the container is brought down onto the measuring instrument stops. In such cases, it takes a further longer time to determine the remaining amount of the medicine packs inside the container.

In the present aspect, the mover moves in synchronization with the change in the length of the medicine packs in the aligned direction of the multiple medicine packs due to the reduction in the remaining amount of the medicine packs inside the container, and the remaining amount of the medicine packs inside the container is determined based on the position of that mover detected by the sensor. Due to such a configuration, the remaining amount of the medicine packs inside the container can be determined simply by detecting the position of the mover that moves in accordance with the reduction in the remaining amount of the medicine packs inside the container. Accordingly, the remaining amount of the medicine packs inside the container can be determined without moving the container, and the length of time required to determine the remaining amount of the medicine packs inside the container can be shortened compared with cases in which the container has to be lifted and brought down onto a measurement device.

### Second Aspect

In the first aspect, the sensor detects at least that the mover has moved to a first position such as a near end position corresponding to a first remaining amount such as the remaining amount in a nearly ended state and that the mover has moved to a second position such as an end position corresponding to a second remaining amount such as zero, i.e., the remaining amount in an ended state, smaller than the first remaining amount.

Due to such a configuration, the remaining amount of the medicine packs inside the container can be figured out at at least two stages.

### Third Aspect

In the second aspect, the sensor is configured to change the first position.

Due to such a configuration, even when the length of a single medicine pack inside a container in the aligned direction of that medicine pack, such as the thickness of a single medicine pack, changes, the first position can appropriately be changed depending on such changes. Accordingly, the remaining amount of the medicine packs inside the container can appropriately be figured out.

### Fourth Aspect

In any one of the first to third aspects, a plurality of containers including the container are further provided, a plurality of movers including the mover are further provided, the mover is disposed on each one of the plurality of containers, the sensor is configured by a single sensor such as the remaining-amount sensor 157, the near-end sensor 157A according to the first modification, and the end sensor 157B according to the first modification to detect a position of each of the plurality of movers disposed on the plurality of containers, and the remaining-amount determination unit determines the remaining amount of the plurality of medicine packs inside each of the plurality of containers based on the detection result of the single sensor.

Due to such a configuration, compared with cases in which the sensor has to be provided for each of the two or more containers, the number of sensors can be reduced to simplify the configuration or structure of the sensors.

### Fifth Aspect

In the fourth aspect, the pickup device is configured to move to a plurality of pack-pickup positions of the plurality of containers to pick up at least one of the plurality of medicine packs from at least one of the plurality of containers, and the single sensor is disposed on the pickup device.

Due to such a configuration, when the pickup device picks up at least one of the medicine packs from each one of the containers, the position of the mover in each one of the containers can be detected by the sensor provided for the pickup device. Accordingly, it is not necessity to move the pickup device or the sensor just to detect the position of the mover in each one of the containers, and the position of the mover in each one of the containers can efficiently be detected.

### Sixth Aspect

In any one of the first to fifth aspects, a calculator such as the controller 150 to calculate an amount of displacement of the mover, such as the thickness T1 of a single medicine pack 2, due to a one-time pickup of one of the plurality of medicine packs, based on the detection result of the sensor, is further provided.

Due to such a configuration, for example, the number of the medicine packs 2 remaining in the container can be obtained with a high degree of precision, and an error that occurs when the medicine pack is picked up from the container by the pickup device can appropriately be dealt with.

### Seventh Aspect

In the sixth aspect, the calculator calculates an amount of displacement of the mover due to the one-time pickup of the one of the plurality of medicine packs, based on a difference between a position of the mover before one of the plurality of medicine packs is taken out from the container by the pickup device and a position of the mover after the one of the plurality of medicine packs is taken out.

Due to such a configuration, an amount of displacement of the mover, such as the thickness T1 of a single medicine pack 2, due to a one-time pickup of one of the plurality of medicine packs, can easily be calculated.

### Eighth Aspect

In the seventh aspect, a controller is further provided that controls the pickup device to retry to pick up one of the plurality of medicine packs when the difference is equal to or smaller than a first predetermined threshold such as the third predetermined value.

Due to such a configuration, it can be figured out that no medicine pack is picked up by a pickup device based on the difference calculated by the calculator, and the pickup device can retry to pick up of medicine packs as desired.

### Ninth Aspect

In the eighth aspect, a notifier is further provided, and when a number of times to retry to pick up one of the plurality of medicine packs reaches a predetermined number of times such as the fourth predetermined value, the controller causes the notifier to report that the number of times to retry to pick up one of the plurality of medicine packs has reached the predetermined number of times.

Due to such a configuration, when a retry is performed a predetermined number of times but no medicine pack is picked up by a pickup device, a user is notified of such a state, and a user is prompted to deal with an error appropriately.

### Tenth Aspect

In any one of the seventh to ninth aspects, a notifier is further provided that report when the difference is equal to or greater than a second predetermined threshold such as the fifth predetermined value.

Due to such a configuration, multiple feeding in which medicine packs have been picked up more than necessary by a pickup device can be detected, and a user is notified of such a state. Accordingly, a user is prompted to deal with an error appropriately.

### Eleventh Aspect

A medication support apparatus includes a medicine-pack housing including a container in which a plurality of medicine packs are stored, a pickup device to pick up at least one of the plurality of medicine packs from the container of the medicine-pack housing, and a conveyor to convey the at least one of the plurality of medicine packs picked up by the pickup device to a medicine tray such as the medicine dispensing tray 30, and the medicine-pack housing according to any one of the first aspect to the tenth aspect is used as the medicine-pack housing.

Due to such a configuration, medication support apparatuses can be provided that determine the remaining amount of the medicine packs inside the container in a short period of time.

The above-described embodiments are illustrative and do not limit the present disclosure. Thus, numerous additional modifications and variations are possible in light of the above teachings. For example, elements and/or features of different illustrative embodiments may be combined with each other and/or substituted for each other within the scope of the present disclosure.

Any one of the above-described operations may be performed in various other ways, for example, in an order different from the one described above.

The functionality of the elements disclosed herein may be implemented using circuitry or processing circuitry which includes general purpose processors, special purpose processors, integrated circuits, application-specific integrated circuits (ASICs), field-programmable gate arrays (FPGAs), and/or combinations thereof which are configured or programmed, using one or more programs stored in one or more memories, to perform the disclosed functionality. Processors are considered processing circuitry or circuitry as they include transistors and other circuitry therein. In the disclosure, the circuitry, units, or means are hardware that carry out or are programmed to perform the recited functionality. The hardware may be any hardware disclosed herein which is programmed or configured to carry out the recited functionality.

There is a memory that stores a computer program which includes computer instructions. These computer instructions provide the logic and routines that enable the hardware (e.g., processing circuitry or circuitry) to perform the method disclosed herein. This computer program can be implemented in known formats as a computer-readable storage medium, a computer program product, a memory device, a record medium such as a compact disc-read-only memory (CD-ROM) or digital versatile disk (DVD), and/or the memory of an FPGA or ASIC.

What is claimed is:

1. A medicine-pack housing comprising:
a container in which a plurality of medicine packs are aligned in line and stored;
a pickup device to pick up at least one of the plurality of medicine packs from the container;
a mover to move in synchronization with a change in a length of the plurality of medicine packs in an aligned direction of the plurality of medicine packs due to a reduction in a remaining amount of the plurality of medicine packs inside the container;
a sensor to detect a position of the mover; and
processing circuitry configured to determine the remaining amount of the plurality of medicine packs inside the container based on a detection result of the sensor.

2. The medicine-pack housing according to claim 1,
wherein the sensor detects at least that the mover has moved to a first position corresponding to a first remaining amount and that the mover has moved to a second position corresponding to a second remaining amount smaller than the first remaining amount.

3. The medicine-pack housing according to claim 2,
wherein the sensor changes the first position.

4. The medicine-pack housing according to claim 1, further comprising:
a plurality of containers including the container; and
a plurality of movers including the mover,
wherein the mover is disposed on each one of the plurality of containers,
wherein the sensor includes a single sensor to detect a position of each of the plurality of movers disposed on the plurality of containers, and
wherein the processing circuitry determines the remaining amount of the plurality of medicine packs inside each of the plurality of containers based on the detection result of the single sensor.

5. The medicine-pack housing according to claim 4,
wherein the pickup device moves to a plurality of pack-pickup positions of the plurality of containers to pick up the at least one of the plurality of medicine packs from at least one of the plurality of containers, and
wherein the single sensor is disposed on the pickup device.

6. The medicine-pack housing according to claim 1,
wherein the processing circuitry calculates an amount of displacement of the mover due to a one-time pickup of one of the plurality of medicine packs, based on the detection result of the sensor.

7. The medicine-pack housing according to claim 6,
wherein the processing circuitry calculates an amount of displacement of the mover due to the one-time pickup of the one of the plurality of medicine packs, based on a difference between a position of the mover before the one of the plurality of medicine packs is taken out from the container by the pickup device and a position of the mover after the one of the plurality of medicine packs is taken out.

8. The medicine-pack housing according to claim 7,
wherein the processing circuitry causes the pickup device to retry to pick up one of the plurality of medicine packs from the container when the difference is equal to or smaller than a threshold.

9. The medicine-pack housing according to claim 8, further comprising:
a notifier,
wherein when a number of times to retry to pick up one of the plurality of medicine packs reaches a predetermined number of times, the processing circuitry causes the notifier to report that the number of times to retry to pick up one of the plurality of medicine packs has reached the predetermined number of times.

10. The medicine-pack housing according to claim 7, further comprising
a notifier,
wherein when the difference is equal to or greater than a threshold, the processing circuitry causes the notifier to report that the difference is equal to or greater than the threshold.

11. A medication support apparatus comprising:
a medicine-pack housing including
a container in which a plurality of medicine packs are aligned in line and stored,
a mover to move in synchronization with a change in a length of the plurality of medicine packs in an aligned direction of the plurality of medicine packs due to a reduction in a remaining amount of the plurality of medicine packs inside the container,
a sensor to detect a position of the mover, and
processing circuitry configured to determine the remaining amount of the plurality of medicine packs inside the container based on a detection result of the sensor;
a pickup device to pick up at least one of the plurality of medicine packs from the container of the medicine-pack housing; and
a conveyor to convey the at least one of the plurality of medicine packs picked up by the pickup device to a medicine tray.
